(19) United States
(12) Patent Application Publication
(10) Pub. No.: US 2014/0163041 A1
(43) Pub. Date: Jun. 12, 2014

(54) LYSINE DEMETHYLASE INHIBITORS FOR MYELOPROLIFERATIVE OR LYMPHOPROLIFERATIVE DISEASES OR DISORDERS

(71) Applicants: Matthew Colin Thor Fyfe, Chipping Norton (GB); Tamara Maes, Castelldefels (ES); Marc Martinell Pedemonte, Barcelona (ES)

(72) Inventors: Matthew Colin Thor Fyfe, Chipping Norton (GB); Tamara Maes, Castelldefels (ES); Marc Martinell Pedemonte, Barcelona (ES)

(73) Assignee: ORYZON GENOMICS S.A., Comella de Llobregat-Barcelona (ES)

(21) Appl. No.: 13/983,844

(22) PCT Filed: Feb. 8, 2012
(86) PCT No.: PCT/EP12/52145

§ 371 Date: Feb. 20, 2014

Related U.S. Application Data
(60) Provisional application No. 61/462,863, filed on Feb. 8, 2011.

(51) Int. Cl. C07D 241/04; C07D 207/14; C07C 311/13; C07C 237/20; A61K 31/145; A61K 31/164; A61K 31/4965; A61K 31/44; A61K 31/40; A61K 31/135; C07D 213/38; C07C 215/64

(52) U.S. Cl. CPC C07D 241/04; C07D 213/38; C07D 207/14; C07C 311/13; C07C 237/20; C07C 215/64; A61K 31/164; A61K 31/4965; A61K 31/44; A61K 31/40; A61K 31/135; A61K 31/145
USPC 514/255.01; 514/357; 514/426; 514/605; 514/619; 514/647

(57) ABSTRACT

The present invention relates to methods and compositions for the treatment or prevention of diseases and disorder associated with myeloproliferative and lymphoproliferative disorders. In particular, the invention relates to an LSD1 inhibitor for use in treating or preventing diseases and disorder associated with myeloproliferative and lymphoproliferative disorders.

### FIELD OF THE INVENTION

The invention relates to methods and compositions for the treatment or prevention of diseases and disorder associated with myeloproliferative and lymphoproliferative disorders. The invention also relates to an LSD1 inhibitor for use in treating or preventing diseases and disorders associated with myeloproliferative and lymphoproliferative disorders.

### BACKGROUND OF THE INVENTION

Myeloproliferative and lymphoproliferative disorders in humans are a major health problem.

Myeloproliferative and lymphoproliferative disorders are characterized as a group of diseases related to abnormal proliferation of blood cells produced in bone marrow.

Myeloproliferative disorders include Philadelphia chromosome positive and Philadelphia chromosome negative categories. Clinically, Philadelphia chromosome positive myeloproliferation is associated with leukemias like chronic myelogenous leukemia (CML) and occasionally in acute myelogenous leukemia (AML) and in related diseases of the lymphoproliferation in respective lineages like acute lymphoblastic leukemia (ALL).

A group of enzymes known as lysine methyl transferases and lysine demethylases are involved in histone lysine modifications. One particular human lysine demethylase enzyme called Lysine Specific Demethylase-1 (LSD1) was recently discovered (Shi et al. (2004) Cell 119:941) and shown to be involved in histone lysine methylation. LSD1 has a fair degree of structural similarity, and amino acid identity/homology to polyamine oxidases and monoamine oxidases, all of which (i.e., MAO-A, MAO-B and LSD1) are flavin dependent amine oxidases which catalyze the oxidation of nitrogen-hydrogen bonds and/or nitrogen-carbon bonds. Although the main target of LSD1 appears to be mono- and di-methylated histone lysines, specifically H3K4 and H3K9, there is evidence in the literature that LSD1 can demethylate methylated lysines on non-histone proteins like p53, E2F1, Dnmt1 and STAT3.

Several groups have reported LSD1 inhibitors in the literature. Sharma et al. recently reported a new series of urea and thiourea analogs based on an earlier series of polyamines which were shown to inhibit LSD1 and modulate histone methylation and gene expression in cells (J. Med. Chem. 2010 PMID: 20568780 [PubMed—as supplied by publisher]). Sharma et al. note that “To date, only a few existing compounds have been shown to inhibit LSD1.” Some efforts were made to make analogs of the histone peptide that is methylated by the enzyme, other efforts have focused on more small molecule like molecules based on known MAO inhibitors. Gooden et al. reported trans-2-arylcyclopropylamine analogues that inhibit LSD1 with Ki values in the range of 188-566 micromolar (Gooden et al. ((2008) Bioorg. Med. Chem. Let. 18:3047-3051)). Most of these compounds were more potent against MAO-A as compared to MAO-B. Ueda et al. ((2009) J. Am. Chem. Soc. 131(48):17536-17537) reported cyclopropylamine analogs selective for LSD1 over MAO-A and MAO-B that were designed based on reported X-ray crystal structures of these enzymes with a phenylcyclopropylamine-FAD adduct and a FAD-N-propargyl lysine peptide; the reported IC50 values for phenylcyclopropylamine were about 32 micromolar for LSD1 whereas compounds 1 and 2 had values of 2.5 and 1.9 micromolar respectively.

Importantly, studies have also been conducted on amine oxidase inhibitor compounds to determine selectivity for MAO-A versus MAO-B since MAO-A inhibitors can cause dangerous side-effects (see e.g., Yoshida et. al. (2004) Bioorg. Med. Chem. 12(10):2645-2652; Hruschka et al. (2008) Biorg Med. Chem. (16):7148-7166; Folks et al. (1983) J. Clin. Psychopharmacol. (3)249; and Youdim et al. (1983) Mod. Probl. Pharmacopsychiatry (19):63).

Currently, the treatments available for myeloproliferative or lymphoproliferative disorders and related diseases have serious drawbacks. There is a need for new drugs for these diseases that target novel points of intervention in the disease processes and avoid side-effects associated with certain targets.

### BRIEF SUMMARY OF THE INVENTION

The present invention relates to the treatment or prevention of hematological cancers, and in particular myeloproliferative disorders or a related disease (e.g., caused by myeloproliferation) or lymphoproliferative disorders or a related disease (e.g., caused by lymphoproliferation). The inventors have unexpectedly found that inhibitors of LSD1 reduce platelets and other blood cells and can therefore be used for the treatment or prevention of myeloproliferative disorders or related diseases or lymphoproliferative disorders or related diseases. The finding was unexpected since LSD1 inhibition is shown to have specific effect on reducing platelets and other blood cells in animal studies. Furthermore, studies by the inventors with myeloproliferative cell lines indicate activity in this setting. Advantageously, the use of selective LSD1 inhibitors or dual LSD1/MAOB inhibitors avoid side-effects associated with targets such as MAOA. The inventors found that administration of LSD1 inhibitors chronically was well tolerated in mammals (selective and dual LSD1/MAOB inhibitors). Thus, the inventors have unexpectedly found that LSD1 inhibition, selective LSD1 inhibition or LSD1/MAOB dual inhibition represent a new therapeutic approach to treating or preventing myeloproliferative disorders or related diseases or lymphoproliferative disorders or related diseases.

The present invention provides for the treatment or prevention of cancer, or a related disease, caused by myeloproliferation. In particular, the invention provides compositions and methods that can be used to reduce platelets or other blood cells and medical benefits derived therefrom.

The present invention provides for the treatment or prevention of cancer, or a related disease, caused by lymphoproliferation. In particular, the invention provides compositions and methods that can be used to reduce lymphocytes or other blood cells and medical benefits derived therefrom.

Thus, the treatment or prevention of a cancer caused by or related to myeloproliferation, comprises administering to an individual in need of treatment or prevention, a therapeutically effective amount of a LSD1 inhibitor. The individual in need of treatment or prevention can be a human or e.g., another mammal. In one aspect, the therapeutically effective amount is an amount sufficient to treat or prevent said cancer. In one aspect, the therapeutically effective amount is an amount sufficient to reduce platelets.

Thus, the treatment or prevention of a cancer caused by or related to lymphoproliferation, comprises administering to an individual in need of treatment or prevention, a therapeutically effective amount of a LSD1 inhibitor. The individual in need of treatment or prevention can be a human or e.g., another mammal. In one aspect, the therapeutically effective amount is an amount sufficient to treat or prevent said cancer. In one aspect, the therapeutically effective amount is an amount sufficient to reduce platelets.

Accordingly, the invention provides for the treatment or prevention methods and compositions based on modulators, particularly inhibitors, of LSD1.

In another embodiment, the invention provides a method of treating or preventing a myeloproliferative disease or disorder in an individual (e.g., a human) by administering a therapeutically effective amount of a LSD1 inhibitor wherein said therapeutically effect amount is an amount sufficient to reduce platelets wherein said disease or disorder is chronic myelogenous leukemia (CML), chronic neutrophilic leukemia, or chronic eosinophilic leukemia. According to one aspect of this embodiment, said method comprises determining whether said individual is Philadelphia chromosome positive or Philadelphia chromosome negative. According to one aspect of this embodiment, said individual is Philadelphia chromosome positive and has chronic myelogenous leukemia. According to one aspect of this embodiment, the LSD1 inhibitor is an irreversible or a reversible amine oxidase inhibitor. In one aspect, the amine oxidase inhibitor is a phenylcyclopropylamine derivative or analog (for example an arylcyclopropylamine derivative or a heteroarylcyclopropylamine derivative), a phenelzine derivative or analog, or a propargylamine derivative or analog.

In another embodiment, the invention provides a method of treating or preventing a lymphoproliferative disease or disorder in an individual (e.g., a human) by administering a therapeutically effective amount of a LSD1 inhibitor wherein said therapeutically effect amount is an amount sufficient to reduce platelets wherein said lymphoproliferative disease is follicular lymphoma, chronic lymphocytic leukemia, acute lymphoblastic leukemia, hairy cell leukemia, lymphoma, multiple myeloma, or Waldenstrom's-macroglobulinemia. According to one aspect of this embodiment, the LSD1 inhibitor is an irreversible or a reversible amine oxidase inhibitor. In one aspect, the amine oxidase inhibitor is a phenylcyclopropylamine derivative or analog (for example an arylcyclopropylamine derivative or a heteroarylcyclopropylamine derivative), a phenelzine derivative or analog, or a propargylamine derivative or analog.

In another embodiment, the invention provides a method of treating or preventing a lymphoproliferative disease or disorder in an individual (e.g., a human) by administering a therapeutically effective amount of a LSD1 inhibitor wherein said therapeutically effect amount is an amount sufficient to reduce platelets wherein said lymphoproliferative disease is multiple myeloma. According to one aspect of this embodiment, the LSD1 inhibitor is an irreversible or a reversible amine oxidase inhibitor. In one aspect, the amine oxidase inhibitor is a phenylcyclopropylamine derivative or analog (for example an arylcyclopropylamine derivative or a heteroarylcyclopropylamine derivative), a phenelzine derivative or analog, or a propargylamine derivative or analog.

In another embodiment, the invention provides a method of treating or preventing a Philadelphia chromosome positive myeloproliferative disease or disorder in an individual (e.g., a human) by administering a therapeutically effective amount of a LSD1 inhibitor wherein said therapeutically effective amount is an amount sufficient to reduce platelets. According to one aspect of this embodiment, the LSD1 inhibitor is an irreversible or a reversible amine oxidase inhibitor. In one aspect, the amine oxidase inhibitor is a phenylcyclopropylamine derivative or analog (for example an arylcyclopropylamine derivative or a heteroarylcyclopropylamine derivative), a phenelzine derivative or analog, or a propargylamine derivative or analog. In one embodiment, the Philadelphia chromosome positive myeloproliferative disease is chronic myelogenous leukemia.

The invention further provides a method of identifying compounds that have activity against myeloproliferation, lymphoproliferation or an associated disease or disorder. More particularly, the method involves identifying a compound that inhibits LSD1 and then testing the LSD1 inhibitors in an assay for myeloproliferation or lymphoproliferation or a related disease or disorder. According to this embodiment an assay system is employed to detect compounds and/or compositions that affect myeloproliferation or lymphoproliferation. In one aspect, said myeloproliferation or lymphoproliferation is modulation of blood cell levels.

The invention, in one embodiment, is a method of treating or preventing a symptom of a Philadelphia chromosome positive myeloproliferative disease in an individual (e.g., a human) having a Philadelphia chromosome positive myeloproliferative disease comprising identifying a patient/individual in need of such treatment or prevention and administering to said individual an amount of a LSD 1 inhibitor sufficient to improve the symptom or reduce the rate of decline of the symptom thereby treating or preventing said symptom. In a related aspect, the invention is the use of a LSD1 inhibitor in an amount sufficient to modulate LSD1 activity for treating or preventing CML, acute myelogenous leukemia (AML), Leukemia stem cells, in an individual having one of these diseases or disorders. In a related aspect, the invention is the use of a LSD1 inhibitor in an amount sufficient to modulate LSD1 activity for treating or preventing CML in an individual having CML. In a related aspect, the invention is the use of a LSD1 inhibitor in an amount sufficient to modulate LSD1 activity for treating or preventing AML in an individual having AML. In one embodiment of this aspect, the method further comprises determining if the individual is Philadelphia chromosome positive or Philadelphia chromosome negative. In one embodiment of this aspect, the method further comprises determining if the individual has a BCR-ABL fusion. In one embodiment of this aspect, the amount of LSD1 inhibitor administered is sufficient to modulate or inhibit LSD1 activity while not substantially inhibiting MAOA activity, thereby avoiding or reducing side-effects associated with administration of MAOA inhibitors.

In one aspect, the invention relates to a pharmaceutical composition for treating or preventing myeloproliferation or lymphoproliferation comprising an anti-myeloproliferative or anti-lymphoproliferative effective amount of a LSD1 inhibitor.

In one aspect, the invention relates to a pharmaceutical composition for treating Philadelphia chromosome positive myeloproliferative disorder comprising a platelet reducing effective amount of a LSD1 inhibitor. In one embodiment, the Philadelphia chromosome positive myeloproliferative disorder is CML.

In one aspect, the invention relates to a method of combination treatment. According to this method a LSD1 inhibitor and a second anti-myeloproliferative or anti-lymphoproliferative agent are administered to an individual (e.g. a human) in need of treatment wherein said individual has a myeloproliferative or lymphoproliferative disease or disorder. In a more specific aspect, the second agent is preferably a kinase inhibitor. In an even more specific aspect, the kinase inhibitor is a BCR-ABL kinase inhibitor. In yet a more specific aspect, the BCR-ABL kinase inhibitor is chosen from imatinib, nilotinib, or dasatinib.

In one aspect, the invention relates to a composition for combination treatment of a myeloproliferative or lymphoproliferative disease. Accordingly, the pharmaceutical composition of this aspect comprises a LSD1 inhibitor and a second anti-myeloproliferative or anti-myeloproliferative agent along with a pharmaceutically acceptable carrier or excipient. In one aspect, the second agent is preferably a BCR-ABL kinase inhibitor.

In one aspect, the invention relates to a composition for combination treatment of a Philadelphia chromosome positive myeloproliferative disease. Accordingly, the pharmaceutical composition of this aspect comprises a LSD1 inhibitor and a second anti-myeloproliferative agent along with a pharmaceutically acceptable carrier or excipient. In one aspect, the second agent is a BCR-ABL kinase inhibitor.

In one aspect, the sufficient period of time for administering the LSD1 inhibitors is from 5 or more days to the individual, more preferably from 5 days to 4 years, even more preferably from 5 days to two years, yet even more preferably for 15 days to 2 years, and again yet even more preferably from 15 days to 1 year. In one aspect, the LSD1 inhibitor is administered daily in amount sufficient to yield a Cmax above the IC50 value for the LSD1 inhibitor. A person skilled in the art will appreciate that the Cmax should be above the IC50 value in the same species (e.g., in a human) in which the Cmax is to be measured.

The invention also relates to an LSD1 inhibitor for use in any of the above-described methods.

Accordingly, the invention relates to a LSD1 inhibitor for use in the treatment or prevention of hematological cancer. The invention also relates to a pharmaceutical composition comprising a LSD1 inhibitor and a pharmaceutically acceptable carrier for use in the treatment or prevention of hematological cancer. In particular, the invention provides a LSD1 inhibitor for use in the treatment or prevention of a hematological cancer caused by or related to myeloproliferation, such as, e.g., acute myelogenous leukemia (AML), chronic myelogenous leukemia (CML), chronic neutrophilic leukemia, or chronic eosinophilic leukemia. The invention thus relates to a LSD1 inhibitor for use in the treatment or prevention of a myeloproliferative disease/disorder or a disease/disorder caused by or related to myeloproliferation. In one aspect, said hematological cancer caused by or related to myeloproliferation is a Philadelphia chromosome positive hematological cancer; accordingly, in one aspect the subject/individual to be treated (e.g., a human) preferably is Philadelphia chromosome positive. The invention also provides a LSD1 inhibitor for use in the treatment or prevention of a hematological cancer caused by or related to lymphoproliferation, such as, e.g., follicular lymphoma, chronic lymphocytic leukemia, acute lymphoblastic leukemia (ALL), hairy cell leukemia, lymphoma, multiple myeloma, or Waldenstrom's macroglobulinemia. The hematological cancer to be treated or prevented may also be a lymphoma chosen from precursor B-lymphoblastic leukemia/lymphoma, B-cell chronic lymphocytic leukemia/small lymphocytic lymphoma, B-cell prolymphocytic leukemia, lymphoplamacytic lymphoma, splenic marginal zone B-cell lymphoma (+/− villous lymphocytes), hairy cell leukemia, plasma cell myeloma/plasmacytoma, extranodal marginal zone B-cell lymphoma of mucosa-associated lymphoid tissue type, nodal marginal zone lymphoma (+/− monocytoid B-cells), follicle center lymphoma, follicular, mantle cell lymphoma, diffuse large cell B-cell lymphoma (mediastinal large B-cell lymphoma or primary effusion lymphoma), Burkitt's lymphoma/Burkitt's cell leukemia, precursor T-lymphoblastic lymphoma/leukemia, T cell prolymphocytic leukemia, T-cell granular lymphocytic leukemia, aggressive NK-Cell leukemia, adult T cell lymphoma/leukemia (HTLV1+), extranodal NK/T-cell lymphoma (nasal type), enteropathy-type T-cell lymphoma, hepatosplenic gamma-delta T-cell lymphoma, subcutaneous panniculitis-like T-cell lymphoma, mycosis fungoides/Sézary's syndrome, anaplastic large cell lymphoma (T/null cell, primary cutaneous type), peripheral T cell lymphoma (not otherwise characterized), angioimmunoblastic T cell lymphoma, anaplastic large cell lymphoma (T/null cell, primary systemic type), nodular lymphocyte predominance Hodgkin's lymphoma, or classical Hodgkin's lymphoma (nodular sclerosis Hodgkin's lymphoma, lymphocyte-rich classical Hodgkin's lymphoma, mixed cellularity Hodgkin's lymphoma or lymphocyte depletion Hodgkin's lymphoma). The invention thus relates to a LSD1 inhibitor for use in the treatment or prevention of a lymphoproliferative disease/disorder or a disease/disorder caused by or related to lymphoproliferation. Furthermore, the present invention provides a LSD1 inhibitor to be administered in combination with one or more further therapeutic agents, in particular an anti-myeloproliferative agent or an anti-lymphoproliferative agent, preferably a kinase inhibitor, more preferably a BCR-ABL kinase inhibitor, and even more preferably imatinib, nilotinib or dasatinib, for use in the treatment or prevention of the above-mentioned therapeutic indications, including the treatment or prevention of hematological cancer. The administration may, e.g., be simultaneous/concomitant or sequential/separate.

The LSD1 inhibitor to be used in accordance with the present invention (e.g., in the treatment or prevention of hematological cancer) is preferably a small molecule inhibitor of LSD1. In a preferred embodiment, the LSD1 inhibitor is a selective LSD1 inhibitor or a dual LSD1/MAO-B inhibitor. In another embodiment, the LSD1 inhibitor is a 2-cyclylcyclopropan-1-amine compound, a phenelzine compound or a propargylamine compound, and is more preferably a 2-cyclylcyclopropan-1-amine compound. Said 2-cyclylcyclopropan-1-amine compound is preferably a 2-arylcyclopropan-1-amine compound or a 2-heteroarylcyclopropan-1-amine compound, more preferably a 2-phenylcyclopropan-1-amine compound, a 2-pyridinylcyclopropan-1-amine compound or a 2-thiazolylcyclopropan-1-amine compound.

Thus the invention particularly relates to the following preferred embodiments:
1. A method of treating or preventing a hematological cancer wherein said hematological cancer is a myeloproliferative disorder or related disease or a lymphoproliferative disorder or a related disease comprising administering to an individual a therapeutically effective amount of a LSD1 inhibitor.
2. The method as in 1 wherein said therapeutically effective amount of a LSD1 inhibitor is an amount sufficient to reduce platelets.
3. The method as in 1 wherein said hematological cancer is CML, AML, or ALL.
4. The method as in 1 wherein said hematological cancer is a Philadelphia chromosome positive myeloproliferative disease or disorder chosen from CML, AML, or ALL.
5. The method as in 1 wherein said LSD1 inhibitor is a selective LSD1 inhibitor.
6. The method as in 1 wherein said LSD1 inhibitor is a dual inhibitor of LSD1 and MAOB.
7. The method as in 1 wherein said LSD1 inhibitor is an irreversible or a reversible amine oxidase inhibitor.
8. The method as in 1 wherein said LSD1 inhibitor is a phenylcyclopropylamine derivative or analog, a phenelzine derivative or analog, or a propargylamine derivative or analog.
9. The method as in 1 wherein said LSD1 inhibitor is a phenylcyclopropylamine derivative or analog.
10. The method as in 1 wherein said LSD1 inhibitor is a phenelzine derivative or analog.
11. The method as in 1 wherein said LSD1 inhibitor is a propargylamine derivative or analog.
12. The method as in 1 further comprising determining if the individual is Philadelphia chromosome positive or Philadelphia chromosome negative.
13. The method as in 1 further comprising determining if the individual is positive for or has a BCR-ABL fusion.
14. The method of claim 1 wherein said hematological cancer is a lymphoma chosen from Precursor B-lymphoblastic leukemia/lymphoma, B-cell chronic lymphocytic leukemia/small lymphocytic lymphoma, B-cell prolymphocytic leukemia, Lymphoplasmacytic lymphoma, Splenic marginal zone B-cell lymphoma (+/− villous lymphocytes), Hairy cell leukemia, Plasma cell myeloma/plasmacytoma, Extranodal marginal zone B-cell lymphoma of mucosa-associated lymphoid tissue type, Nodal marginal zone lymphoma (+/− monocytoid B-cells), Follicle center lymphoma, follicular, Mantle cell lymphoma Diffuse large cell B-cell lymphoma (Mediastinal large B-cell lymphoma or Primary effusion lymphoma), Burkitt's lymphoma/Burkitt's cell leukemia, Precursor T-lymphoblastic lymphoma/leukemia, T cell prolymphocytic leukemia, T-cell granular lymphocytic leukemia, Aggressive NK-Cell leukemia, Adult T cell lymphoma/leukemia (HTLV1+), Extranodal NK/T-cell lymphoma (nasal type), Enteropathy-type T-cell lymphoma, Hepatosplenic gamma-delta T-cell lymphoma, Subcutaneous panniculitis-like T-cell lymphoma, Mycosis fungoides/Sézary's syndrome, Anaplastic large cell lymphoma (T/null cell, primary cutaneous type), Peripheral T cell lymphoma (not otherwise characterized), Angioimmunoblastic T cell lymphoma, and Anaplastic large cell lymphoma (T/null cell, primary systemic type), Nodular lymphocyte predominance Hodgkin's lymphoma, or Classical Hodgkin's lymphoma (Nodular sclerosis Hodgkin's lymphoma, Lymphocyte-rich classical Hodgkin's lymphoma, Mixed cellularity Hodgkin's lymphoma or Lymphocyte depletion Hodgkin's lymphoma).
15. The method of claim 1 wherein said hematological cancer is a lymphoma chosen from follicular lymphoma, chronic lymphocytic leukemia, acute lymphoblastic leukemia, hairy cell leukemia, lymphoma, multiple myeloma, or Waldenstrom's macroglobulinemia.
16. The method of claim 1 wherein said hematological cancer is multiple myeloma.
17. The method as in claim 1 further comprising determining if the individual is positive for or has one or more of the following B-cell lymphoma markers: CD5, CD10, CD19, CD20, CD21, CD22, CD23, CD43, CD79a, sIg, or cIg.
18. The method as in claim 1 further comprising determining if the individual is positive for or has one or more of the following T-cell lymphoma markers: CD3, CD5, CD7, CD4, CD8, CD30, or NK16/56.
19. The method as in 1 further comprising administering second anti-myeloproliferative or anti-lymphoproliferative agent to said individual.
20. The method as in 1 further comprising administering a second anti-myeloproliferative or anti-lymphoproliferative agent to said individual wherein said second myeloproliferative agent is chosen from imatinib, nilotinib, or dasatinib.
21. A Pharmaceutical composition comprising a LSD1 inhibitor and a pharmaceutically acceptable carrier for use in any one of 1-16.
22. The LSD1 inhibitor of 17 wherein said LSD1 inhibitor is a selective LSD1 inhibitor.
23. The LSD1 inhibitor of 17 wherein said LSD1 inhibitor is a dual inhibitor of LSD1 and MAOB.
24. The LSD1 inhibitor of 17 wherein said LSD1 inhibitor is an irreversible or a reversible amine oxidase inhibitor.
25. The LSD1 inhibitor of 17 wherein said LSD1 inhibitor is a phenylcyclopropylamine derivative or analog, a phenelzine derivative or analog, or a propargylamine derivative or analog.
26. The LSD1 inhibitor of 17 wherein said LSD1 inhibitor is a phenylcyclopropylamine derivative or analog.
27. The LSD1 inhibitor of 17 wherein said LSD1 inhibitor is a phenelzine derivative or analog.
28. The LSD1 inhibitor of 17 wherein said LSD1 inhibitor is a propargylamine derivative or analog.

### BRIEF DESCRIPTION OF THE DRAWINGS

FIG. 1 Optimization of Selective LSD1 Inhibitors. FIG. 1 summarizes structure-activity relationship evolution of increased potency towards LSD1 as compared to MAOA and/or MAOB from compounds that were not selective (e.g., tranylcypromine (TCPA)) to compounds that are selective inhibitors of LSD1 with IC50 values in the low nanomolar range.

FIG. 2 Optimization of Dual LSD1/MAOB Inhibitors. FIG. 2 summarizes structure-activity relationship evolution of increased potency towards LSD1 and MAOB as compared to MAOA from compounds that were not selective for LSD1 and MAOB (e.g., tranylcypromine (TCPA)). The dual LSD1/MAOB compounds have IC50 values for these two targets in the low nanomolar range.

FIG. 3 Compound Dual-1 Increases Histone Methylation. FIG. 3 shows the results of a western blot stained for H3K4 methylation with SH-SY5Y cells grown in the presence of Compound Dual-1 (at 100 μM) or parnate (“PNT”) (at 250 μM) for 1, 2, and 3 days, showing that this compound, Dual-1, increases H3K4 methylation in cells in a time dependent manner.

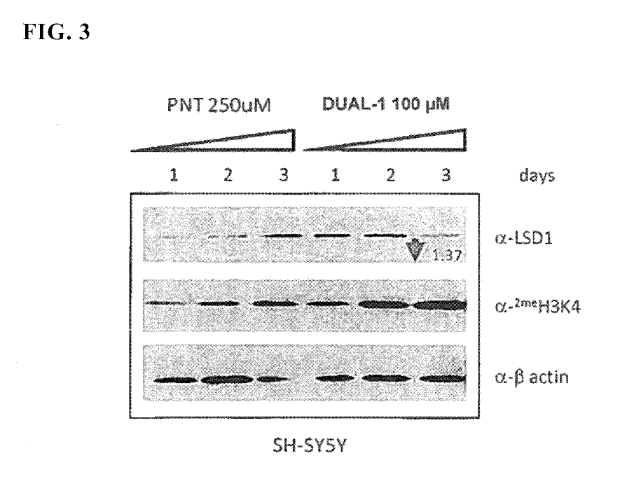

### DETAILED DESCRIPTION OF THE INVENTION

The present invention relates to the treatment or prevention of hematological cancers, and in particular myeloproliferative disorders or related disease (e.g., caused by myeloproliferation) or lymphoproliferative disorders or a related disease (e.g., caused by lymphoproliferation). The inventors have unexpectedly found that inhibitors of LSD1 reduce platelets and other blood cells and can therefore be used for the treatment or prevention of myeloproliferative disorders or related diseases or lymphoproliferative disorders or related diseases. The finding was unexpected since LSD1 inhibition is shown to have specific effect on reducing platelets and other blood cells in animal studies. Thus, the methods and compositions of the invention can be useful for treating myeloproliferative or lymphoproliferative disorders where the individual is resistant to or not effectively treated by current medications or that cannot comply with the treatment regimes employed with current medications. Additionally, the methods and compositions of the present invention can be useful for treating or preventing Philadelphia chromosome positive myeloproliferation in combination with other anti-myeloproliferative agents or anti-lymphoproliferative agents used in this clinical setting. Other advantages and more details of the invention are described below.

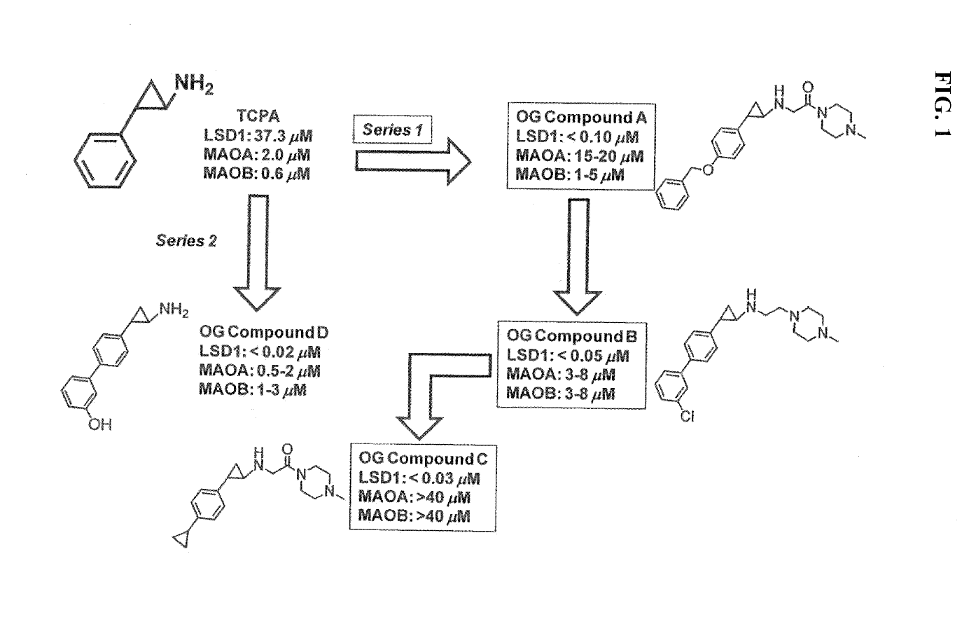

A medicinal chemistry effort undertaken by some of the inventors resulted in the synthesis and identification of small molecule, potent selective LSD1 inhibitors and potent dual inhibitors of LSD1 and MAOB. This effort resulted in the identification of a number of compounds having different selectivities for LSD1, MAOA, and MAOB. See FIG. 1.

Subsequent studies of some of the optimized compounds in a neural derived cell line and other cell lines indicted that both selective LSD1 inhibitors and dual inhibitors of LSD1 and MAOB can increase histone methylation levels at the cellular level indicating that these compounds inhibit cellular lysine demethylase activity. Furthermore, these LSD1 inhibitors show dose dependent effects on gene expression levels in these cells.

Lastly the LSD1 inhibitors were to be able to be administered to mammals chronically at doses that are thought to achieve levels of the inhibitor sufficient for causing a biological effect.

As a result of these studies, a number of LSD1 inhibitors were shown to have activity in reducing platelets and other blood cells in vivo. Without being bound by theory, it is believed that LSD1 inhibitors, including 2-cyclylcyclopropan-1-amine compounds, phenelzine compounds, propargylamine compounds and other LSD1 inhibitors, inhibit platelet and blood cell proliferation and have use for treating or preventing hematological cancers associated with a myeloproliferative or lymphoproliferative disorder or a related disease. More specifically, it is believed that LSD1 inhibitors, as a result of this invention, have use in treating or preventing cancers like myeloma, leukemia, or lymphoma.

### Methods of Treatment or Prevention and Disease

The invention relates to methods of treatment or prevention of a hematological cancer related to myeloproliferation or lymphoproliferation with LSD1 inhibitors, and pharmaceutical compositions for treating or preventing myeloproliferation or lymphoproliferation.

The present invention provides for the treatment or prevention of cancer, or a related disease, caused by or related to myeloproliferation. In particular, the invention provides compositions and methods that can be used to reduce platelets or other blood cells and medical benefits derived therefrom.

The present invention provides for the treatment or prevention of cancer, or a related disease, caused by or related to lymphoproliferation. In particular, the invention provides compositions and methods that can be used to reduce platelets or other blood cells and medical benefits derived therefrom.

In one embodiment, the invention is the use of a LSD1 inhibitor for treating or preventing a hematological cancer related to or caused by myeloproliferation. In one aspect of this embodiment, said hematological cancer is chronic myelogenous leukemia (CML), chronic neutrophilic leukemia, or chronic eosinophilic leukemia. In a related aspect, the invention is a method of treating or preventing Philadelphia chromosome positive myeloproliferation comprising administering a LSD1 inhibitor to an individual. In another related aspect, the invention is a method of treating or preventing Philadelphia chromosome positive myeloproliferation comprising administering a LSD1 inhibitor to an individual in need of such treatment. In yet another related aspect, the invention is a method of treating or preventing Philadelphia chromosome positive myeloproliferation comprising identifying an individual in need of such treatment or prevention and administering a LSD1 inhibitor to said individual. In a related aspect, the invention is the use of a LSD1 inhibitor in an amount sufficient to modulate LSD1 activity for treating or preventing CML in an individual having CML. In a related aspect, the invention is the use of a LSD1 inhibitor in an amount sufficient to modulate LSD1 activity for treating or preventing chronic neutrophilic leukemia in an individual having chronic neutrophilic leukemia. In a related aspect, the invention is the use of a LSD1 inhibitor in an amount sufficient to modulate LSD1 activity for treating or preventing chronic eosinophilic leukemia in an individual having chronic eosinophilic leukemia. In one aspect of the method described in this paragraph, the method further comprises determining if the individual is Philadelphia chromosome positive or Philadelphia chromosome negative. In one aspect of the method described in this paragraph, the method further comprises determining if the individual has a BCR-ABL fusion. In one aspect, the LSD1 inhibitor described in this paragraph is a small molecule inhibitor of LSD1. In one aspect, the LSD1 inhibitor described in this paragraph is a selective inhibitor of LSD1. In one aspect, the LSD1 inhibitor described in this paragraph is a selective inhibitor of LSD1 and MAOB. In one aspect, the LSD1 inhibitor described in this paragraph is an irreversible or a reversible amine oxidase inhibitor. In one aspect, the amine oxidase inhibitor of this paragraph is a phenylcyclopropylamine derivative or analog, a phenelzine derivative or analog, or a propargylamine derivative or analog. In one aspect, the LSD1 inhibitor described in this paragraph is a 2-cyclylcyclopropan-1-amine compound, a phenelzine compound, or a propargylamine compound, more preferably a 2-cyclylcyclopropan-1-amine compound, still more preferably a 2-arylcyclopropan-1-amine compound or a 2-heteroarylcyclopropan-1-amine compound, and even more preferably a 2-phenylcyclopropan-1-amine compound, a 2-pyridinylcyclopropan-1-amine compound or a 2-thiazolylcyclopropan-1-amine compound.

In one embodiment, the invention is the use of an amount of an LSD1 inhibitor sufficient for reducing platelets for inhibiting myeloproliferation in a patient having a hematological cancer. In an aspect, the invention is a method of inhibiting myeloproliferation comprising administering a LSD1 inhibitor to an individual in an amount sufficient to reduce platelets. In another related aspect, the invention is a method of inhibiting myeloproliferation comprising administering a LSD1 inhibitor in an amount sufficient to reduce platelets to an individual in need of such treatment. In yet another related aspect, the invention is a method of inhibiting Philadelphia chromosome positive myeloproliferation comprising identifying an individual in need of such treatment or prevention and administering a LSD1 inhibitor, in an amount sufficient to reduce platelets, to said individual. In a related aspect, the invention is the use of a LSD1 inhibitor, in an amount sufficient to reduce platelets, for treating or preventing CML in an individual having CML. In a related aspect, the invention is the use of a LSD1 inhibitor, in an amount sufficient to reduce platelets, for treating or preventing chronic neutrophilic leukemia in an individual having chronic neutrophilic leukemia. In a related aspect, the invention is the use of a LSD1 inhibitor, in an amount sufficient to reduce platelets, for treating or preventing chronic eosinophilic leukemia in an individual having chronic eosinophilic leukemia. In one aspect of the method described in this paragraph, the method further comprises determining if the individual is Philadelphia chromosome positive or Philadelphia chromosome negative. In one aspect of the method described in this paragraph, the method further comprises determining if the individual has a BCR-ABL fusion. In one aspect, the LSD1 inhibitor described in this paragraph is a small molecule inhibitor of LSD1. In one aspect, the LSD1 inhibitor described in this paragraph is a selective inhibitor of LSD1. In one aspect, the LSD1 inhibitor described in this paragraph is a selective inhibitor of LSD1 and MAOB. In one aspect, the LSD1 inhibitor described in this paragraph is an irreversible or a reversible amine oxidase inhibitor. In one aspect, the amine oxidase inhibitor of this paragraph is a phenylcyclopropylamine derivative or analog, a phenelzine derivative or analog, or a propargylamine derivative or analog. In one aspect, the LSD1 inhibitor described in this paragraph is a 2-cyclylcyclopropan-1-amine compound, a phenelzine compound, or a propargylamine compound, more preferably a 2-cyclylcyclopropan-1-amine compound, still more preferably a 2-arylcyclopropan-1-amine compound or a 2-heteroarylcyclopropan-1-amine compound, and even more preferably a 2-phenylcyclopropan-1-amine compound, a 2-pyridinylcyclopropan-1-amine compound or a 2-thiazolylcyclopropan-1-amine compound.

In one embodiment, the invention is the use of a LSD1 inhibitor for treating or preventing a hematological cancer related to or caused by lymphoproliferation. In one aspect of this embodiment, said hematological cancer is follicular lymphoma, chronic lymphocytic leukemia, acute lymphoblastic leukemia, hairy cell leukemia, lymphoma, multiple myeloma, and Waldenstrom's macroglobulinemia. In a related aspect, the invention is a method of treating or preventing lymphoproliferation comprising administering a LSD1 inhibitor to an individual. In another related aspect, the invention is a method of treating or preventing a hematological cancer related to or caused by lymphoproliferation comprising administering a therapeutically effective amount of a LSD1 inhibitor to an individual in need of such treatment. In yet another related aspect, the invention is a method of treating or preventing a hematological cancer related to or caused by lymphoproliferation comprising identifying an individual in need of such treatment or prevention and administering a LSD1 inhibitor to said individual. In a related aspect, the invention is the use of a LSD1 inhibitor in an amount sufficient to modulate LSD1 activity for treating or preventing follicular lymphoma in an individual having follicular lymphoma. In a related aspect, the invention is the use of a LSD1 inhibitor in an amount sufficient to modulate LSD1 activity for treating or preventing chronic lymphocytic leukemia in an individual having chronic lymphocytic leukemia. In a related aspect, the invention is the use of a LSD1 inhibitor in an amount sufficient to modulate LSD1 activity for treating or preventing acute lymphoblastic leukemia in an individual having acute lymphoblastic leukemia. In a related aspect, the invention is the use of a LSD1 inhibitor in an amount sufficient to modulate LSD1 activity for treating or preventing hairy cell leukemia in an individual having hairy cell leukemia. In a related aspect, the invention is the use of a LSD1 inhibitor in an amount sufficient to modulate LSD1 activity for treating or preventing lymphoma in an individual having lymphoma. In a related aspect, the invention is the use of a LSD1 inhibitor in an amount sufficient to modulate LSD1 activity for treating or preventing multiple myeloma in an individual having multiple myeloma. In a related aspect, the invention is the use of a LSD1 inhibitor in an amount sufficient to modulate LSD1 activity for treating or preventing Waldenstrom's macroglobulinemia in an individual having Waldenstrom's macroglobulinemia. In one aspect of the method described in this paragraph, the method further comprises determining if the individual is Philadelphia chromosome positive or Philadelphia chromosome negative or another marker of a lymphoproliferative disease or cancer. In one aspect of the method described in this paragraph, the method further comprises determining if the individual has a BCR-ABL fusion. In one aspect, the LSD1 inhibitor described in this paragraph is a small molecule inhibitor of LSD1. In one aspect, the LSD1 inhibitor described in this paragraph is a selective inhibitor of LSD1. In one aspect, the LSD1 inhibitor described in this paragraph is a selective inhibitor of LSD1 and MAOB. In one aspect, the LSD1 inhibitor described in this paragraph is an irreversible or a reversible amine oxidase inhibitor. In one aspect, the amine oxidase inhibitor of this paragraph is a phenylcyclopropylamine derivative or analog, a phenelzine derivative or analog, or a propargylamine derivative or analog. In one aspect, the LSD1 inhibitor described in this paragraph is a 2-cyclylcyclopropan-1-amine compound, a phenelzine compound, or a propargylamine compound, more preferably a 2-cyclylcyclopropan-1-amine compound, still more preferably a 2-arylcyclopropan-1-amine compound or a 2-heteroarylcyclopropan-1-amine compound, and even more preferably a 2-phenylcyclopropan-1-amine compound, a 2-pyridinylcyclopropan-1-amine compound or a 2-thiazolylcyclopropan-1-amine compound.

In one embodiment, the invention is the use of a LSD1 inhibitor for treating or preventing a lymphoma. In a related aspect, the invention is a method of treating or preventing lymphoma comprising administering a therapeutically effective amount of a LSD1 inhibitor to an individual having lymphoma wherein said lymphoma is chosen from Precursor B-lymphoblastic leukemia/lymphoma, B-cell chronic lymphocytic leukemia/small lymphocytic lymphoma, B-cell prolymphocytic leukemia, Lymphoplasmacytic lymphoma, Splenic marginal zone B-cell lymphoma (+/− villous lymphocytes), Hairy cell leukemia, Plasma cell myeloma/plasmacytoma, Extranodal marginal zone B-cell lymphoma of mucosa-associated lymphoid tissue type, Nodal marginal zone lymphoma (+/− monocytoid B-cells), Follicle center lymphoma, follicular, Mantle cell lymphoma Diffuse large cell. B-cell lymphoma (Mediastinal large B-cell lymphoma or Primary effusion lymphoma), Burkitt's lymphoma/Burkitt's cell leukemia, Precursor T-lymphoblastic lymphoma/leukemia, T cell prolymphocytic leukemia, T-cell granular lymphocytic leukemia, Aggressive NK-Cell leukemia, Adult T cell lymphoma/leukemia (HTLV 1+), Extranodal NK/T-cell lymphoma (nasal type), Enteropathy-type T-cell lymphoma, Hepatosplenic gamma-delta T-cell lymphoma, Subcutaneous panniculitis-like T-cell lymphoma, Mycosis fungoides/Sézary's syndrome, Anaplastic large cell lymphoma, T/null cell, primary cutaneous type, Peripheral T cell lymphoma, not otherwise characterized, Angioimmunoblastic T cell lymphoma, and Anaplastic large cell lymphoma, T/null cell, primary systemic type, Nodular lymphocyte predominance Hodgkin's lymphoma, Classical Hodgkin's lymphoma (Nodular sclerosis Hodgkin's lymphoma, Lymphocyte-rich classical Hodgkin's lymphoma, Mixed cellularity Hodgkin's lymphoma or Lymphocyte depletion Hodgkin's lymphoma). In another related aspect, the invention is a method of treating or preventing lymphoma comprising administering a LSD1 inhibitor to an individual in need of such treatment wherein said individual has a marker for a lymphoma. In yet another related aspect, the invention is a method of treating or preventing lymphoma comprising identifying an individual having a lymphoma marker and is in need of such treatment or prevention and administering a LSD1 inhibitor to said individual. In one embodiment of this aspect, the method further comprises determining if the individual has one or more of the following B-cell lymphoma markers: CD5, CD10, CD19, CD20, CD21, CD22, CD23, CD43, CD79a, or sIg cIg. In one embodiment of this aspect, the method further comprises determining if the individual has one or more of the following T-cell lymphoma markers: CD3, CD5, CD7, CD4, CD8, CD30, or NK16/56. In one aspect, the LSD1 inhibitor described in this paragraph is a small molecule inhibitor of LSD1. In one aspect, the LSD1 inhibitor is a selective inhibitor of LSD1. In one aspect, the LSD1 inhibitor is a selective inhibitor of LSD1 and MAOB. In one aspect, the LSD1 inhibitor is an irreversible or a reversible amine oxidase inhibitor. In one aspect, the irreversible amine oxidase inhibitor is a phenylcyclopropylamine derivative or analog, a phenelzine derivative or analog, or a propargylamine derivative or analog. In one aspect, the LSD1 inhibitor described in this paragraph is a 2-cyclylcyclopropan-1-amine compound, a phenelzine compound, or a propargylamine compound, more preferably a 2-cyclylcyclopropan-1-amine compound, still more preferably a 2-arylcyclopropan-1-amine compound or a 2-heteroarylcyclopropan-1-amine compound, and even more preferably a 2-phenylcyclopropan-1-amine compound, a 2-pyridinylcyclopropan-1-amine compound or a 2-thiazolylcyclopropan-1-amine compound.

The patient, subject, or individual, such as the individual in need of treatment or prevention, may be e.g. a eukaryote, an animal, a vertebrate animal, a mammal, a rodent (e.g. a guinea pig, a hamster, a rat, a mouse), a murine (e.g. a mouse), a canine (e.g. a dog), a feline (e.g. a cat), an equine (e.g. a horse), a primate, a simian (e.g. a monkey or ape), a monkey (e.g. a marmoset, a baboon), an ape (e.g. gorilla, chimpanzee, orangutang, gibbon), or a human. The meaning of the terms “eukaryote”, “animal”, “mammal”, etc. is well known in the art and can, for example, be deduced from Wehner and Gehring (1995; Thieme Verlag). In the context of this invention, it is particularly envisaged that animals are to be treated which are economically, agronomically or scientifically important. Scientifically important organisms include, but are not limited to, mice, rats, rabbits, fruit flies like Drosophila melagonaster and nematodes like Caenorhabditis elegans. Non-limiting examples of agronomically important animals are sheep, cattle and pig, while, for example, cats and dogs may be considered as economically important animals. Preferably, the subject/patient/individual is a mammal; more preferably, the subject/patient/individual is a human.

As used herein, the term “treating a disease or disorder” refers to a slowing of or a reversal of the progress of the disease. Treating a disease or disorder includes treating a symptom and/or reducing the symptoms of the disease.

As used herein, the term “preventing a disease or disorder” refers to a slowing of the disease or of the onset of the disease or the symptoms thereof. Preventing a disease or disorder can include stopping the onset of the disease or symptoms thereof.

As used herein, “LSD1 inhibitor” refers to a molecule that directly or indirectly lowers or downregulates a biological activity of Lysine Dependent Demethylase 1 (LSD1). A LSD1 inhibitor may be any member of a class of compounds (e.g. a small molecule or an antibody) that binds LSD1 and inhibits a biological activity (e.g. demethylase activity) of a LSD1 protein or a protein complex in which LSD1 exerts its function (e.g. LSD1 being complexed to co-REST and/or other protein members of the nucleosome). A LSD1 inhibitor may also be any member of a class of compounds that decreases the expression of a nucleic acid encoding a LSD1 protein (e.g. an inhibitory nucleic acid, RNAi, such as a small hairpin RNA). Preferably, a LSD1 inhibitor is a compound that exhibits LSD1-inhibitory activity in the LSD1 biological assay disclosed in Example 1. The skilled person is able to determine whether a compound would qualify as LSD1 inhibitor in such assay. Preferably, a LSD1 inhibitor is a compound that exhibits more than 50% inhibition of LSD1 activity in the LSD1 assay of example 1 at 50 mcM, more preferably one that exhibits more than 50% inhibition of LSD1 activity in the LSD1 assay of example 1 at 10 mcM, still more preferably one that exhibits more than 50% inhibition of LSD1 activity in the LSD1 assay of example 1 at 1 mcM, and even more preferably one that exhibits more than 50% inhibition of LSD1 activity in the LSD1 assay of example 1 at a concentration of 0.5 mcM or less.

As used herein “a small molecule inhibitor of LSD1” refers to an LSD1 inhibitor having a molecular weight of less than 1.000 daltons, preferably less than 700 daltons.

As used herein, the term “selective LSD1 inhibitor”, “LSD1 selective inhibitor” or “selective inhibitor of LSD1” refers to an LSD1 inhibitor which preferably has an 1050 value for LSD1 that is at least two-fold lower than its 1050 values for MAO-A and MAO-B. More preferably, a selective LSD1 inhibitor has an IC50 value for LSD1 which is at least five-fold lower than its IC50 values for MAO-A and MAO-B. Even more preferably, a selective LSD1 inhibitor has an IC50 value for LSD1 which is at least ten-fold lower than its IC50 values for MAO-A and MAO-B. Even more preferably, a selective LSD1 inhibitor has an IC50 value for LSD1 which is at least 20-fold lower than its 1050 values for MAO-A and MAO-B. Even more preferably, a selective LSD1 inhibitor has an 1050 value for LSD1 which is at least 50-fold lower than its IC50 values for MAO-A and MAO-B. Even more preferably, a selective LSD1 inhibitor has an 1050 value for LSD1 which is at least 100-fold lower than its 1050 values for MAO-A and MAO-B. The ability of a compound to inhibit LSD1 and its 1050 values for LSD1, MAO-A and MAO-B are preferably to be determined in accordance with the experimental protocol described in Example 1.

As used herein, the term “selective inhibitor of LSD1 and MAOB”, “dual LSD1/MAO-B inhibitor”, “LSD1/MAO-B inhibitor”, “dual LSD1/MAOB selective inhibitor”, “dual inhibitor selective for LSD1 and MAO-B” or “dual inhibitor of LSD1 and MAO-B” are used interchangeably and refers to an LSD1 inhibitor which preferably has 1050 values for LSD1 and MAO-B which are at least two-fold lower than its 1050 value for MAO-A. More preferably, a dual LSD1/MAO-B selective inhibitor has 1050 values for LSD1 and MAO-B which are at least five-fold lower than its IC50 value for MAO-A. Even more preferably, a dual LSD1/MAO-B selective inhibitor has 1050 values for LSD1 and MAO-B which are at least ten-fold lower than its IC50 value for MAO-A. Even more preferably, a dual LSD1/MAO-B selective inhibitor has IC50 values for LSD1 and MAO-B which are at least 20-fold lower than its IC50 value for MAO-A. The ability of a compound to inhibit LSD1 and MAO-B and its IC50 values for LSD1, MAO-A and MAO-B are preferably to be determined in accordance with the experimental protocol described in Example 1.

In context of this invention, a “reduction in platelets (or other blood cells)” or a “reduction of platelet (or other blood cells) levels” may, accordingly, comprise the reduction in platelet/cell count. As illustrated in the appended examples, the compounds of the present invention are surpassingly capable of reducing cell count/cell levels, in particular of blood cells and most particular of platelets. Accordingly, the LSD1 inhibitors as provided herein are useful in reducing (blood) cell counts/levels, in particular in reducing counts/levels of platelets. A “reduction in count/level” in this respect can be measured by means and methods provided herein and in the appended examples. A “reduction in (blood) cell and/or platelet levels” and/or a “reduction (blood) cell and/or platelet counts” can comprise the measurement of a given biological samples, like a blood sample, derived from a patient in need of medical intervention as provided herein in comparison to a given control sample or control samples or as compared to standard references or standard reference values. Such a control sample or such control samples may comprise corresponding samples from healthy individuals or from defined diseased individuals (for example individuals suffering from or being prone to suffer from hematological cancers like myeloproliferative or lymphoproliferative disorders. Such a control sample may also comprise a biological sample from the same individual to be assessed (like the patient) whereby said sample was taken at an earlier or a later stage when said individual was or is healthy or diseased (i.e. before, during or after medical intervention as disclosed herein). In context of this invention the “platelet reduction” to be achieved with the compounds of the present invention is a reduction of at least 10%, at least 20%, at least 30% or more as compared to a control sample or as compared to standard references or standard reference values.”

As used herein, the term “unit dosage form” refers to a physically discrete unit, such as a capsule or tablet suitable as a unitary dosage for a human patient. Each unit contains a predetermined quantity of a LSD1 inhibitor, which was discovered or believed to produce the desired pharmacokinetic profile which yields the desired therapeutic effect. The dosage unit is composed of a LSD1 inhibitor in association with at least one pharmaceutically acceptable carrier, salt, excipient, or combination thereof.

In another aspect, the invention is a method of treating or preventing Philadelphia chromosome positive myeloproliferation comprising identifying an individual in need of such treatment or prevention and administering to said individual for a sufficient period of time an amount of a LSD1 inhibitor, preferably a selective LSD1 inhibitor, sufficient to treat or prevent Philadelphia chromosome positive myeloproliferation. In a related aspect, the invention is the use of a LSD1 inhibitor, preferably a selective LSD1 inhibitor, in an amount sufficient to modulate LSD1 activity for treating or preventing Philadelphia chromosome positive myeloproliferation. In a specific aspect, said treatment reduces Philadelphia chromosome positive myeloproliferation. In one embodiment of this aspect, the amount of LSD1 inhibitor, preferably a selective LSD1 inhibitor, administered is sufficient to modulate or inhibit LSD1 activity while not substantially inhibiting MAOA activity, thereby avoiding or reducing side-effects associated with administration of MAOA inhibitors. In a specific aspect of this embodiment, preferably the amount of LSD1 inhibitor administered per day to a human is from about 0.5 mg to about 500 mg per day. More preferably the amount of LSD1 inhibitor administered per day to a human is from about 0.5 mg to about 200 mg per day or is a pharmaceutical composition formulated in such a way as to deliver this amount of free base equivalent (or free acid equivalent depending on the parent molecule). Preferably, the LSD1 inhibitor is administered or formulated to be administered for 5 or more days to the individual, more preferably from 5 days to 4 years, even more preferably from 5 days to two years, yet even more preferably for 15 days to 2 years, and again yet even more preferably from 15 days to 1 year. It is noted that in this context administration for e.g., 5 or more days, means an amount sufficient over a time sufficient to cause pharmacologic inhibition of LSD1 over this period of time and this does not necessarily mean administration of compound every day or only once per day. Depending on the PK/ADME properties of the inhibitors, a suitable amount and dosing regimen can be determined by a skilled practitioner in view of this disclosure.

In one aspect, the invention is a method of treating or preventing Philadelphia chromosome positive myeloproliferation comprising identifying an individual in need of such treatment or prevention and administering to said individual for a sufficient period of time an amount of a dual LSD1/MAOB inhibitor sufficient to treat or prevent Philadelphia chromosome positive myeloproliferation. In a related aspect, the invention is the use of a dual LSD1/MAOB inhibitor in an amount sufficient to modulate Philadelphia chromosome positive myeloproliferative activity for treating or preventing Philadelphia chromosome positive myeloproliferation. In a specific aspect, treating or preventing Philadelphia chromosome positive myeloproliferation comprises reducing platelets. In one embodiment of this aspect, the amount of a dual LSD1/MAOB inhibitor administered is sufficient to modulate or inhibit LSD1 and MAOB activity while not substantially inhibiting MAOA activity, thereby avoiding or reducing side-effects associated with administration of MAOA inhibitors. In a specific aspect of this embodiment, preferably the amount of dual LSD1/MAOB inhibitor administered per day to a human is from about 0.5 mg to about 500 mg per day. More preferably the amount of dual LSD1/MAOB inhibitor administered per day to a human is from about 0.5 mg to about 200 mg per day or is a pharmaceutical composition formulated in such a way as to deliver this amount of free base equivalent (or free acid equivalent depending on the parent molecule). In one embodiment of this aspect, the amount of dual LSD1/MAOB inhibitor administered is sufficient to modulate or inhibit LSD1/MAOB activity while not substantially inhibiting MAO-A activity, thereby avoiding or reducing side-effects associated with administration of MAO-A inhibitors. Preferably, the dual LSD1/MAOB inhibitor is administered or formulated to be administered for 5 or more days to the individual, more preferably from 5 days to 4 years, even more preferably from 5 days to two years, yet even more preferably for 15 days to 2 years, and again yet even more preferably from 15 days to 1 year. It is noted that in this context administration for e.g., 5 or more days, means an amount sufficient over a time sufficient to cause pharmacologic inhibition of LSD1 and MAOB over this period of time and this does not necessarily mean administration of compound every day or only once per day. Depending on the PK/ADME properties of the inhibitors, a suitable amount and dosing regimen can be determined by a skilled practitioner in view of this disclosure.

In one embodiment, the invention is a method of treating or preventing Philadelphia chromosome positive myeloproliferation comprising identifying an individual in need of such treatment or prevention and administering to said individual a LSD1 inhibitor and a second anti-myeloproliferation agent to treat or prevent Philadelphia chromosome positive myeloproliferation. In a related aspect, the invention is the use of a LSD1 inhibitor and a second anti-myeloproliferation agent in an amount sufficient for treating or preventing Philadelphia chromosome positive myeloproliferation. In a specific aspect, treating or preventing Philadelphia chromosome positive myeloproliferation comprises inhibiting platelets via LSD1 and inhibiting myeloproliferation with a second anti-myeloproliferation agent such as a kinase inhibitor. In one embodiment of this aspect, the amount of second anti-myeloproliferation agent is sufficient to prevent or treat Philadelphia chromosome positive myeloproliferation. In one embodiment of this aspect, the amount of second anti-myeloproliferation agent administered is sufficient to prevent or treat Philadelphia chromosome positive myeloproliferation while avoiding or reducing side-effects associated with administration of higher doses of said second anti-myeloproliferation agent. In one aspect, the second anti-myeloproliferation agent is a BCR-ABL kinase inhibitor. In one aspect, the BCR-ABL kinase inhibitor is chosen from imatinib, nilotinib, or dasatinib. In one aspect, the second anti-myeloproliferative agent is imatinib. In one aspect, the second anti-myeloproliferative agent is nilotinib. In one aspect, the second anti-myeloproliferative agent is dasatinib. In a specific aspect of this embodiment, preferably the amount of LSD1 inhibitor administered per day to a human is from about 0.5 mg to about 500 mg per day. More preferably the amount of LSD1 inhibitor administered per day to a human is from about 0.5 mg to about 200 mg per day or is a pharmaceutical composition formulated in such a way as to deliver this amount of free base equivalent (or free acid equivalent depending on the parent molecule). In one embodiment of this aspect, the amount of the second anti-myeloproliferation agent administered to the individual is from 0.050 to 1000 mg daily. More preferably, the amount of the second anti-myeloproliferation agent is administered to the individual is from 0.050 to 500 mg daily. Even more preferably, the amount of the second anti-myeloproliferation agent administered to the individual is from 0.050 to 200 mg daily. Depending on the PK/ADME properties of the inhibitors, a suitable amount and dosing regimen can be determined by a skilled practitioner in view of this disclosure.

The invention also relates to an LSD1 inhibitor for use in any of the above-described methods.

Accordingly, the invention relates to an LSD1 inhibitor (or a pharmaceutical composition comprising an LSD1 inhibitor and a pharmaceutically acceptable carrier) for use in treating or preventing a hematological cancer. In one embodiment, the hematological cancer is a myeloproliferative disorder or a disease/disorder caused by or related to myeloproliferation or said hematological cancer is a lymphoproliferative disorder or a disease/disorder caused by or related to lymphoproliferation. In a specific embodiment, the hematological cancer is a myeloproliferative disorder or a disease/disorder caused by or related to myeloproliferation. In another specific embodiment, said hematological cancer is a Philadelphia chromosome positive myeloproliferative disease. In a more specific embodiment, the hematological cancer is acute myelogenous leukemia (AML), chronic myelogenous leukemia (CML), chronic neutrophilic leukemia, or chronic eosinophilic leukemia. In another specific embodiment, the hematological cancer is hematological cancer is a hematological cancer caused by or related to lymphoproliferation. In a more specific embodiment, hematological cancer is follicular lymphoma, chronic lymphocytic leukemia (CLL), acute lymphoblastic leukemia (ALL), hairy cell leukemia, lymphoma, multiple myeloma, or Waldenstrom's macroglobulinemia. In another specific embodiment, said hematological cancer is a lymphoma chosen from precursor B-lymphoblastic leukemia/lymphoma, B-cell chronic lymphocytic leukemia/small lymphocytic lymphoma, B-cell prolymphocytic leukemia, lymphoplasmacytic lymphoma, splenic marginal zone B-cell lymphoma (+/− villous lymphocytes), hairy cell leukemia, plasma cell myeloma/plasmacytoma, extranodal marginal zone B-cell lymphoma of mucosa-associated lymphoid tissue type, nodal marginal zone lymphoma (+/− monocytoid B-cells), follicle center lymphoma, follicular, mantle cell lymphoma, diffuse large cell B-cell lymphoma (mediastinal large B-cell lymphoma or primary effusion lymphoma), Burkitt's lymphoma/Burkitt's cell leukemia, precursor T-lymphoblastic lymphoma/leukemia, T cell prolymphocytic leukemia, T-cell granular lymphocytic leukemia, aggressive NK-Cell leukemia, adult T cell lymphoma/leukemia (HTLV1+), extranodal NK/T-cell lymphoma (nasal type), enteropathy-type T-cell lymphoma, hepatosplenic gamma-delta T-cell lymphoma, subcutaneous panniculitis-like T-cell lymphoma, mycosis fungoides/Sézary's syndrome, anaplastic large cell lymphoma (T/null cell, primary cutaneous type), peripheral T cell lymphoma (not otherwise characterized), angioimmunoblastic T cell lymphoma, anaplastic large cell lymphoma (T/null cell, primary systemic type), nodular lymphocyte predominance Hodgkin's lymphoma, or classical Hodgkin's lymphoma (nodular sclerosis Hodgkin's lymphoma, lymphocyte-rich classical Hodgkin's lymphoma, mixed cellularity Hodgkin's lymphoma or lymphocyte depletion Hodgkin's lymphoma). In another specific embodiment, said hematological cancer is multiple myeloma. In one aspect, the LSD1 inhibitor is a small molecule inhibitor of LSD1. In one aspect, the LSD1 inhibitor is a selective inhibitor of LSD1. In one aspect, the LSD1 inhibitor is a selective inhibitor of LSD1 and MAOB (i.e. a dual LSD1/MAO-B inhibitor). In one aspect, the LSD1 inhibitor is a 2-cyclylcyclopropan-1-amine compound, a phenelzine compound, or a propargylamine compound, more preferably a 2-cyclylcyclopropan-1-amine compound, still more preferably a 2-arylcyclopropan-1-amine compound or a 2-heteroarylcyclopropan-1-amine compound, and even more preferably a 2-phenylcyclopropan-1-amine compound, a 2-pyridinylcyclopropan-1-amine compound or a 2-thiazolylcyclopropan-1-amine compound.

Accordingly, the invention relates to an LSD1 inhibitor (or a pharmaceutical composition comprising an LSD1 inhibitor and a pharmaceutically acceptable carrier) for use in treating or preventing a hematological cancer in an individual (e.g. in a human), wherein the LSD1 inhibitor is administered at an amount sufficient to reduce platelet levels in said individual. In one embodiment, the hematological cancer is a myeloproliferative disorder or a disease/disorder caused by or related to myeloproliferation or said hematological cancer is a lymphoproliferative disorder or a disease/disorder caused by or related to lymphoproliferation. In a specific embodiment, the hematological cancer is a myeloproliferative disorder or a disease/disorder caused by or related to myeloproliferation. In another specific embodiment, said hematological cancer is a Philadelphia chromosome positive myeloproliferative disease. In a more specific embodiment, the hematological cancer is acute myelogenous leukemia (AML), chronic myelogenous leukemia (CML), chronic neutrophilic leukemia, or chronic eosinophilic leukemia. In another specific embodiment, the hematological cancer is hematological cancer is a hematological cancer caused by or related to lymphoproliferation. In a more specific embodiment, hematological cancer is follicular lymphoma, chronic lymphocytic leukemia (CLL), acute lymphoblastic leukemia (ALL), hairy cell leukemia, lymphoma, multiple myeloma, or Waldenstrom's macroglobulinemia. In another specific embodiment, said hematological cancer is a lymphoma chosen from precursor B-lymphoblastic leukemia/lymphoma, B-cell chronic lymphocytic leukemia/small lymphocytic lymphoma, B-cell prolymphocytic leukemia, lymphoplasmacytic lymphoma, splenic marginal zone B-cell lymphoma (+/− villous lymphocytes), hairy cell leukemia, plasma cell myeloma/plasmacytoma, extranodal marginal zone B-cell lymphoma of mucosa-associated lymphoid tissue type, nodal marginal zone lymphoma (+/− monocytoid B-cells), follicle center lymphoma, follicular, mantle cell lymphoma, diffuse large cell B-cell lymphoma (mediastinal large B-cell lymphoma or primary effusion lymphoma), Burkitt's lymphoma/Burkitt's cell leukemia, precursor T-lymphoblastic lymphoma/leukemia, T cell prolymphocytic leukemia, T-cell granular lymphocytic leukemia, aggressive NK-Cell leukemia, adult T cell lymphoma/leukemia (HTLV1+), extranodal NK/T-cell lymphoma (nasal type), enteropathy-type T-cell lymphoma, hepatosplenic gamma-delta T-cell lymphoma, subcutaneous panniculitis-like T-cell lymphoma, mycosis fungoides/Sézary's syndrome, anaplastic large cell lymphoma (T/null cell, primary cutaneous type), peripheral T cell lymphoma (not otherwise characterized), angioimmunoblastic T cell lymphoma, anaplastic large cell lymphoma (T/null cell, primary systemic type), nodular lymphocyte predominance Hodgkin's lymphoma, or classical Hodgkin's lymphoma (nodular sclerosis Hodgkin's lymphoma, lymphocyte-rich classical Hodgkin's lymphoma, mixed cellularity Hodgkin's lymphoma or lymphocyte depletion Hodgkin's lymphoma). In another specific embodiment, said hematological cancer is multiple myeloma. In one aspect, the LSD1 inhibitor is a small molecule inhibitor of LSD1. In one aspect, the LSD1 inhibitor is a selective inhibitor of LSD1. In one aspect, the LSD1 inhibitor is a selective inhibitor of LSD1 and MAOB (i.e. a dual LSD1/MAO-B inhibitor). In one aspect, the LSD1 inhibitor is a 2-cyclylcyclopropan-1-amine compound, a phenelzine compound, or a propargylamine compound, more preferably a 2-cyclylcyclopropan-1-amine compound, still more preferably a 2-arylcyclopropan-1-amine compound or a 2-heteroarylcyclopropan-1-amine compound, and even more preferably a 2-phenylcyclopropan-1-amine compound, a 2-pyridinylcyclopropan-1-amine compound or a 2-thiazolylcyclopropan-1-amine compound.

Accordingly, the invention relates to an LSD1 inhibitor (or a pharmaceutical composition comprising an LSD1 inhibitor and a pharmaceutically acceptable carrier) for use in treating or preventing a symptom of a hematological cancer. In one embodiment, the hematological cancer is a myeloproliferative disorder or a disease/disorder caused by or related to myeloproliferation or said hematological cancer is a lymphoproliferative disorder or a disease/disorder caused by or related to lymphoproliferation. In a specific embodiment, the hematological cancer is a myeloproliferative disorder or a disease/disorder caused by or related to myeloproliferation. In another specific embodiment, said hematological cancer is a Philadelphia chromosome positive myeloproliferative disease. In a more specific embodiment, the hematological cancer is acute myelogenous leukemia (AML), chronic myelogenous leukemia (CML), chronic neutrophilic leukemia, or chronic eosinophilic leukemia. In another specific embodiment, the hematological cancer is hematological cancer is a hematological cancer caused by or related to lymphoproliferation. In a more specific embodiment, hematological cancer is follicular lymphoma, chronic lymphocytic leukemia (CLL), acute lymphoblastic leukemia (ALL), hairy cell leukemia, lymphoma, multiple myeloma, or Waldenstrom's macroglobulinemia. In another specific embodiment, said hematological cancer is a lymphoma chosen from precursor B-lymphoblastic leukemia/lymphoma, B-cell chronic lymphocytic leukemia/small lymphocytic lymphoma, B-cell prolymphocytic leukemia, lymphoplasmacytic lymphoma, splenic marginal zone B-cell lymphoma (+/− villous lymphocytes), hairy cell leukemia, plasma cell myeloma/plasmacytoma, extranodal marginal zone B-cell lymphoma of mucosa-associated lymphoid tissue type, nodal marginal zone lymphoma (+/− monocytoid B-cells), follicle center lymphoma, follicular, mantle cell lymphoma, diffuse large cell B-cell lymphoma (mediastinal large B-cell lymphoma or primary effusion lymphoma), Burkitt's lymphoma/Burkitt's cell leukemia, precursor T-lymphoblastic lymphoma/leukemia, T cell prolymphocytic leukemia, T-cell granular lymphocytic leukemia, aggressive NK-Cell leukemia, adult T cell lymphoma/leukemia (HTLV1+), extranodal NK/T-cell lymphoma (nasal type), enteropathy-type T-cell lymphoma, hepatosplenic gamma-delta T-cell lymphoma, subcutaneous panniculitis-like T-cell lymphoma, mycosis fungoides/Sézary's syndrome, anaplastic large cell lymphoma (T/null cell, primary cutaneous type), peripheral T cell lymphoma (not otherwise characterized), angioimmunoblastic T cell lymphoma, anaplastic large cell lymphoma (T/null cell, primary systemic type), nodular lymphocyte predominance Hodgkin's lymphoma, or classical Hodgkin's lymphoma (nodular sclerosis Hodgkin's lymphoma, lymphocyte-rich classical Hodgkin's lymphoma, mixed cellularity Hodgkin's lymphoma or lymphocyte depletion Hodgkin's lymphoma). In another specific embodiment, said hematological cancer is multiple myeloma. In one aspect, the LSD1 inhibitor is a small molecule inhibitor of LSD1. In one aspect, the LSD1 inhibitor is a selective inhibitor of LSD1. In one aspect, the LSD1 inhibitor is a selective inhibitor of LSD1 and MAOB (i.e. a dual LSD1/MAO-B inhibitor). In one aspect, the LSD1 inhibitor is a 2-cyclylcyclopropan-1-amine compound, a phenelzine compound, or a propargylamine compound, more preferably a 2-cyclylcyclopropan-1-amine compound, still more preferably a 2-arylcyclopropan-1-amine compound or a 2-heteroarylcyclopropan-1-amine compound, and even more preferably 2-phenylcyclopropan-1-amine compound, a 2-pyridinylcyclopropan-1-amine compound or a 2-thiazolylcyclopropan-1-amine compound.

Accordingly, the invention relates to an LSD1 inhibitor (or a pharmaceutical composition comprising an LSD1 inhibitor and a pharmaceutically acceptable carrier) and one or more further therapeutic agents for use in treating or preventing a hematological cancer. In one embodiment, the hematological cancer is a myeloproliferative disorder or a disease/disorder caused by or related to myeloproliferation or said hematological cancer is a lymphoproliferative disorder or a disease/disorder caused by or related to lymphoproliferation. In a specific embodiment, the hematological cancer is a myeloproliferative disorder or a disease/disorder caused by or related to myeloproliferation. In another specific embodiment, said hematological cancer is a Philadelphia chromosome positive myeloproliferative disease. In a more specific embodiment, the hematological cancer is acute myelogenous leukemia (AML), chronic myelogenous leukemia (CML), chronic neutrophilic leukemia, or chronic eosinophilic leukemia. In another specific embodiment, the hematological cancer is hematological cancer is a hematological cancer caused by or related to lymphoproliferation. In a more specific embodiment, hematological cancer is follicular lymphoma, chronic lymphocytic leukemia (CLL), acute lymphoblastic leukemia (ALL), hairy cell leukemia, lymphoma, multiple myeloma, or Waldenstrom's macroglobulinemia. In another specific embodiment, said hematological cancer is a lymphoma chosen from precursor B-lymphoblastic leukemia/lymphoma, B-cell chronic lymphocytic leukemia/small lymphocytic lymphoma, B-cell prolymphocytic leukemia, lymphoplasmacytic lymphoma, splenic marginal zone B-cell lymphoma (+/− villous lymphocytes), hairy cell leukemia, plasma cell myeloma/plasmacytoma, extranodal marginal zone B-cell lymphoma of mucosa-associated lymphoid tissue type, nodal marginal zone lymphoma (+/− monocytoid B-cells), follicle center lymphoma, follicular, mantle cell lymphoma, diffuse large cell B-cell lymphoma (mediastinal large B-cell lymphoma or primary effusion lymphoma), Burkitt's lymphoma/Burkitt's cell leukemia, precursor T-lymphoblastic lymphoma/leukemia, T cell prolymphocytic leukemia, T-cell granular lymphocytic leukemia, aggressive NK-Cell leukemia, adult T cell lymphoma/leukemia (HTLV1+), extranodal NK/T-cell lymphoma (nasal type), enteropathy-type T-cell lymphoma, hepatosplenic gamma-delta T-cell lymphoma, subcutaneous panniculitis-like T-cell lymphoma, mycosis fungoides/Sézary's syndrome, anaplastic large cell lymphoma (T/null cell, primary cutaneous type), peripheral T cell lymphoma (not otherwise characterized), angioimmunoblastic T cell lymphoma, anaplastic large cell lymphoma (T/null cell, primary systemic type), nodular lymphocyte predominance Hodgkin's lymphoma, or classical Hodgkin's lymphoma (nodular sclerosis Hodgkin's lymphoma, lymphocyte-rich classical Hodgkin's lymphoma, mixed cellularity Hodgkin's lymphoma or lymphocyte depletion Hodgkin's lymphoma). In another specific embodiment, said hematological cancer is multiple myeloma. In one aspect, the LSD1 inhibitor is a small molecule inhibitor of LSD1. In one aspect, the LSD1 inhibitor is a selective inhibitor of LSD1. In one aspect, the LSD1 inhibitor is a selective inhibitor of LSD1 and MAOB (i.e. a dual LSD1/MAO-B inhibitor). In one aspect, the LSD1 inhibitor is a 2-cyclylcyclopropan-1-amine compound, a phenelzine compound, or a propargylamine compound, more preferably a 2-cyclylcyclopropan-1-amine compound, still more preferably a 2-arylcyclopropan-1-amine compound or a 2-heteroarylcyclopropan-1-amine compound, and even more preferably a 2-phenylcyclopropan-1-amine compound, a 2-pyridinylcyclopropan-1-amine compound or a 2-thiazolylcyclopropan-1-amine compound. In one embodiment, the further therapeutic agent is an anti-myeloproliferative agent or an anti-lymphoproliferative agent, preferably a kinase inhibitor, more preferably a BCR-ABL kinase inhibitor, and even more preferably imatinib, nilotinib or dasatinib. The administration may, e.g., be simultaneous/concomitant or sequential/separate.

### Compounds, Formulation, and Routes of Administration

The LSD1 inhibitors, selective LSD1 inhibitors and dual LSD1/MAOB inhibitors for use in the invention can be synthesized by a number of techniques including the ones that are described below.

Examples of selective LSD1 and LSD1/MAOB dual inhibitors are given in e.g., WO2010/043721 (PCT/EP2009/063685), WO2010/084160 (PCT/EP2010/050697), WO2011/035941 (PCT/EP2010/055131); WO2011/042217 (PCT/EP2010/055103); WO2012/013727 (PCT/EP2011/062947); WO2011/131697 (PCT/EP2011/056279); WO2012/013728 (PCT/EP2011/062949); PCT/EP2011/067608; and EP applications number EP10171345 (EP10171345.1), EP10187039 (EP10187039.2) and EP10171342 (EP10171342.8) all of which are explicitly incorporated herein by reference in their entireties to the extent they are not inconsistent with the instant disclosure.

In one specific aspect, a phenylcyclopropylamine derivative or analog for use in the invention is phenylcyclopropylamine (PCPA) with one or two substitutions on the amine group; phenylcyclopropylamine with zero, one or two substitutions on the amine group and one, two, three, four, or five substitution on the phenyl group; phenylcyclopropylamine with one, two, three, four, or five substitution on the phenyl group; phenylcyclopropylamine with zero, one or two substitutions on the amine group wherein the phenyl group of PCPA is substituted with (exchanged for) another ring system chosen from aryl or heterocyclyl or heteroaryl to give an aryl- or heterocyclyl- or heteroaryl-cyclopropylamine having zero, one or two substituents on the amine group; phenylcyclopropylamine wherein the phenyl group of PCPA is substituted with (exchanged for) another ring system chosen from aryl or heterocyclyl to give an aryl- or heterocycyl-cyclopropylamine wherein said aryl- or heterocyclyl-cyclopropylamine on said aryl or heterocyclyl moiety has zero, one or two substitutions on the amine group and one, two, three, four, or five substitution on the phenyl group; phenylcyclopropylamine with one, two, three, four, or five substitution on the phenyl group; or any of the above described phenylcyclopropylamine analogs or derivatives wherein the cyclopropyl has one, two, three or four additional substituents. Preferably, the heterocyclyl group described above in this paragraph is a heteroaryl.

Other examples of arylcyclopropylamine derivatives and analogues for use in the invention include those disclosed in, WO2010/143582 (PCT/JP2010/059476), US 2010/0324147 (Ser. No. 12/792,316), S. Mimasu et al. Biochemistry (2010), 49(30):6494-503, C. Binda et al, J. Am. Chem. Soc. (2010), 132(19): 6827-33, D. M. Gooden et al. Bioorg. Med. Chem. Let. (2008) 18:3047-3051, R. Ueda et al. J. Am. Chem. Soc. (2009) 131(48):17536-17537, WO 2011/131576, all of which are explicitly incorporated herein by reference in their entireties to the extent they are not inconsistent with the instant disclosure.

Other examples of LSD1 inhibitors are e.g., phenelzine or pargyline (propargylamine) or a derivative or analog thereof. Derivatives and analogs of phenelzine and pargyline (propargylamine) include, but are not limited to, compounds where the phenyl group of the parent compound is replaced with a heteroaryl or optionally substituted cyclic group or the phenyl group of the parent compound is optionally substituted with a cyclic group and have the selective LSD1 or dual LSD1/MAOB inhibitory activity as described herein. In one aspect, the phenelzine derivative or analog has one, two, three, four or five substituents on the phenyl group. In one aspect, the phenelzine derivative or analog has the phenyl group substituted with (exchanged for) an aryl or heterocyclyl group wherein said aryl or heterocyclyl group has zero, one, two, three, four or five substituents. In one aspect, the pargyline derivative or analog has one, two, three, four or five substituents on the phenyl group. In one aspect, the pargyline derivative or analog has the phenyl group substituted with (exchanged for) an aryl or heterocyclyl group wherein said aryl or heterocyclyl group has zero, one, two, three, four or five substituents. Methods of preparing such compounds are known to the skilled artisan.

The LSD1 inhibitor to be used in accordance with the present invention (e.g., in the treatment or prevention of hematological cancer) is preferably a 2-cyclylcyclopropan-1-amine compound, a phenelzine compound or a propargylamine compound, and is more preferably a 2-cyclylcyclopropan-1-amine compound. Said 2-cyclylcyclopropan-1-amine compound is preferably a 2-arylcyclopropan-1-amine compound or a 2-heteroarylcyclopropan-1-amine compound, more preferably a 2-phenylcyclopropan-1-amine compound, a 2-pyridinylcyclopropan-1-amine compound or a 2-thiazolylcyclopropan-1-amine compound.

It is particularly preferred that the LSD1 inhibitor or selective LSD1 inhibitor or dual LSD1/MAO-B inhibitor is a 2-cyclylcyclopropan-1-amine compound which is a compound of the following formula (I) or an enantiomer, a diastereomer or a mixture of stereoisomers (such as a racemic mixture or a diastereomer mixture) thereof, or a pharmaceutically acceptable salt or solvate thereof:

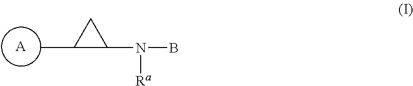

A is cyclyl optionally having 1, 2, 3 or 4 substituents A′. Preferably, said cyclyl is aryl or heteroaryl. Said aryl is preferably phenyl. Said heteroaryl is preferably selected from pyridinyl, pyrimidinyl, thiophenyl, benzothiophenyl, pyrrolyl, indolyl, furanyl or thiazolyl, more preferably said heteroaryl is selected from pyridinyl, pyrimidinyl or thiazolyl, still more preferably said heteroaryl is pyridinyl (in particular, pyridin-2-yl or pyridin-3-yl) or thiazolyl (in particular thiazol-5-yl) and even more preferably said heteroaryl is pyridin-3-yl or thiazol-5-yl.

It is preferred that said cyclyl (or said aryl or said heteroaryl, or any of the above-mentioned specific aryl or heteroaryl groups) is unsubstituted or has 1 or 2 substituents A′, and it is more preferred that said cyclyl (or said aryl or said heteroaryl, or any of the above-mentioned specific aryl or heteroaryl groups) is unsubstituted or has 1 substituent A′.

Said substituent(s) A′ is/are each independently selected from -L¹-cyclyl (e.g., -L¹-aryl, -L¹-cycloalkyl or -L¹-heterocyclyl), alkyl, alkenyl, alkynyl, alkoxy, amino, amido (e.g., —CH₂—CO—NH₂, alkylamino, hydroxyl, nitro, halo, haloalkyl, haloalkoxy, cyano, sulfonyl, sulfinyl, sulfonamide, acyl, carboxyl, carbamate or urea, wherein the cyclyl moiety comprised in said -L¹-cyclyl is optionally further substituted with one or more (e.g., 1, 2 or 3) groups independently selected from halo, haloalkyl, haloalkoxy, aryl, arylalkoxy, aryloxy, arylalkyl, alkyl, alkenyl, alkynyl, alkoxy, amino, amido (e.g., —CO—NH₂), alkylamino, hydroxyl, nitro, —CH₂—CO—NH₂, heteroaryl, heteroarylalkoxy, heteroaryloxy, heteroarylalkyl, cyano, sulfonyl, sulfinyl, sulfonamide, acyl, carboxyl, carbamate or urea, preferably selected from halo, haloalkyl, hydroxy, N-sulfonamido or cyano. It is preferred that the cyclyl moiety comprised in said -L¹-cyclyl is unsubstituted or is substituted with one of the above groups (including, e.g., one of the preferred groups halo, haloalkyl, hydroxy, N-sulfonamido or cyano). In one preferred embodiment, the cyclyl moiety comprised in said -L¹-cyclyl is substituted with one of the above groups (including, e.g., one of the preferred groups halo, haloalkyl, hydroxy, N-sulfonamido or cyano). In another preferred embodiment the cyclyl moiety is unsubstituted. Said -L¹-cyclyl is preferably -L¹-aryl, -L¹-cycloalkyl or -L¹-heterocyclyl (e.g., -L¹-heteroaryl or -L¹-heterocycloalkyl), more preferably -L¹-aryl or -L¹-heteroaryl, even more preferably -L¹-aryl, even more preferably -L¹-phenyl.

Each L¹ is independently selected from a covalent bond, —(CH₂)₁₋₆—, —(CH₂)₀₋₃—O—(CH₂)₀₋₃—, —(CH₂)₀₋₃—NH—(CH₂)₀₋₃— or —(CH₂)₀₋₃—S—(CH₂)₀₋₃—, preferably from a covalent bond, —(CH₂)₁₋₃—, —O—(CH₂)₀₋₃— or —NH—(CH₂)₀₋₃—, more preferably from a covalent bond, —CH₂—, —O—, —O—CH₂—, —O—(CH₂)₂—, —NH— or —NH—CH₂—, even more preferably from a covalent bond, —CH₂— or —O—CH₂—. It is furthermore preferred that the aforementioned groups L¹ (connecting the moiety A to the cyclyl moiety comprised in -L¹-cyclyl) are in the specific orientation indicated above (accordingly, the group “—O—CH₂-” as an example for L¹ is preferably in the orientation ( . . . )-A-O—CH₂-cyclyl).

Preferably, said substituent(s) A′ is/are each independently selected from -L¹-aryl, -L¹-cycloalkyl, -L¹-heteroaryl or -L¹-heterocycloalkyl, wherein said aryl, said cycloalkyl, said heteroaryl or said heterocycloalkyl is optionally substituted with halo (e.g., —F or —Cl), haloalkyl (e.g., —CF₃), hydroxy, N-sulfonamido (e.g. —NHSO₂-aryl, wherein the aryl group can be optionally substituted) or cyano. More preferably, said substituent(s) A′ is/are each independently -L¹-aryl (e.g., -L¹-phenyl), wherein the aryl moiety in said -L¹-aryl (or the phenyl moiety in said -L¹-phenyl) is optionally substituted with halo (e.g., —F or —Cl), haloalkyl (e.g., —CF₃), hydroxy, N-sulfonamido (e.g. —NHSO₂-aryl, wherein the aryl group can be optionally substituted) or cyano. Even more preferably, said substituent(s) A′ is/are each independently phenyl, —CH₂-phenyl, —O—CH₂-phenyl, —NH—CH₂-phenyl or —O—(CH₂)₂-phenyl, wherein said phenyl or the phenyl moiety in said —CH₂-phenyl, said —O—CH₂-phenyl, said —NH—CH₂-phenyl or said —O—(CH₂)₂-phenyl is optionally substituted with halo (e.g., —F or —Cl), haloalkyl (e.g., —CF₃), hydroxy, N-sulfonamido (e.g. —NHSO₂-aryl, wherein the aryl group can be optionally substituted) or cyano. Even more preferably, said substituent(s) A′ is/are each independently phenyl, —CH₂-phenyl, —O—CH₂-phenyl, or —O—(CH₂)₂-phenyl, wherein said phenyl or the phenyl moiety in said —CH₂-phenyl, said —O—CH₂-phenyl or said —O—(CH₂)₂-phenyl is optionally substituted with halo (e.g., —F or —Cl), haloalkyl (e.g., —CF₃), hydroxy, N-sulfonamido (e.g. —NHSO₂-aryl, wherein the aryl group can be optionally substituted) or cyano. Even more preferably, said substituent(s) A′ is/are each independently phenyl, —CH₂-phenyl, or —O—CH₂-phenyl, wherein said phenyl or the phenyl moiety in said —CH₂-phenyl or said —O—CH₂-phenyl is optionally substituted with halo (e.g., —F or —Cl) or haloalkyl (e.g., —CF₃).

It is particularly preferred that A is aryl (preferably phenyl) or heteroaryl (preferably pyridinyl or thiazolyl), which aryl or heteroaryl optionally has one substituent A′ selected from -L¹-aryl, -L¹-cycloalkyl, -L¹-heteroaryl or -L¹-heterocycloalkyl (wherein the aryl moiety in said L¹-aryl, the cycloalkyl moiety in said -L¹-cycloalkyl, the heteroaryl moiety in said -L¹-heteroaryl or the heterocycloalkyl moiety in said -L¹-heterocycloalkyl may be substituted with halo (e.g., —F or —Cl), haloalkyl (e.g., —CF₃), hydroxy, N-sulfonamido or cyano), preferably selected from phenyl, —CH₂-phenyl or —O—CH₂-phenyl (wherein said phenyl, the phenyl moiety in said —CH₂-phenyl or the phenyl moiety in said —O—CH₂-phenyl may be substituted with halo (e.g., —F or —Cl), haloalkyl (e.g., —CF₃)), hydroxy, N-sulfonamido or cyano) and even more preferably selected from phenyl, —CH₂-phenyl or —O—CH₂-phenyl (wherein said phenyl, the phenyl moiety in said —CH₂-phenyl or the phenyl moiety in said —O—CH₂-phenyl may be substituted with halo (e.g., —F or —Cl) or haloalkyl (e.g., —CF₃)).

R^{a} is —H or alkyl. Preferably R^{a} is —H or (C1-C4)alkyl (such as methyl or ethyl), and more preferably R^{a} is —H.

B is -L²-cyclyl, —H, -L²-CO—NH₂, -L²-CO—NR¹R², or -L²-CO—R³, wherein the cyclyl moiety in said -L²-cyclyl is optionally substituted with one or more (e.g., one, two or three) groups independently selected from halo, haloalkyl, haloalkoxy, haloaryl, aryl, arylalkoxy, aryloxy, arylalkyl, alkyl, alkenyl, alkynyl, alkoxy, amino, amido (e.g., —CO—NH₂), alkylamino, hydroxyl, nitro, —CH₂—CO—NH₂, heteroaryl, heteroarylalkoxy, heteroaryloxy, heteroarylalkyl, cycloalkyl, cycloalkylalkoxy, cycloalkoxy, cycloalkylalkyl, heterocycloalkyl, heterocycloalkylalkoxy, heterocycloalkoxy, heterocycloalkylalkyl, cyano, cyanato, isocyanato, thiocyanato, isothiocyanato, sulfonyl, sulfinyl, sulfonamide, trihalomethanesulfonamido, acyl, acylamino, acyloxy, alkylthio, cycloalkylthio, heterocycloalkylthio, arylthio, heteroarylthio, carboxyl, carbamate or urea, preferably selected from halo, alkyl, alkoxy, haloalkyl, haloalkoxy, cyano, hydroxyl, amino, alkylamino, aminoalkyl, amido (e.g., —CO—NH₂), —CH₂—CO—NH₂, or sulfonamide.

It is preferred that the cyclyl moiety in said -L²-cyclyl is unsubstituted or is substituted with one group selected from halo, haloalkyl, haloalkoxy, haloaryl, aryl, arylalkoxy, aryloxy, arylalkyl, alkyl, alkenyl, alkynyl, alkoxy, amino, amido (e.g., —CO—NH₂), alkylamino, hydroxyl, nitro, —CH₂—CO—NH₂, heteroaryl, heteroarylalkoxy, heteroaryloxy, heteroarylalkyl, cycloalkyl, cycloalkylalkoxy, cycloalkoxy, cycloalkylalkyl, heterocycloalkyl, heterocycloalkylalkoxy, heterocycloalkoxy, heterocycloalkylalkyl, cyano, cyanato, isocyanato, thiocyanato, isothiocyanato, sulfonyl, sulfinyl, sulfonamide, trihalomethanesulfonamido, acyl, acylamino, acyloxy, alkylthio, cycloalkylthio, heterocycloalkylthio, arylthio, heteroarylthio, carboxyl, carbamate or urea, preferably selected from halo, alkyl, alkoxy, haloalkyl, haloalkoxy, cyano, hydroxyl, amino, alkylamino, aminoalkyl, amido (e.g., —CO—NH₂), —CH₂—CO—NH₂, or sulfonamide.

The cyclyl moiety in said -L²-cyclyl, which may be substituted as defined and described above, is preferably selected from aryl, cycloalkyl or heterocyclyl (e.g., heteroaryl or heterocycloalkyl), more preferably heterocyclyl, even more preferably from heteroaryl or heterocycloalkyl. Said heteroaryl is preferably selected from oxadiazolyl, thiazolyl or pyrimidinyl. Said heterocycloalkyl is preferably selected from pyrrolidinyl, piperidinyl, piperazinyl, N-methylpiperazinyl or morpholinyl.

In formula (I), R¹ and R² are each independently chosen from —H, alkyl, alkynyl, alkenyl, -L-carbocyclyl, -L-aryl, or -L-heterocyclyl, wherein said alkyl, said alkynyl or said alkenyl is optionally substituted with one or more groups independently selected from halo, haloalkoxy, haloaryl, aryl, arylalkoxy, aryloxy, alkoxy, amino, amido, alkylamino, hydroxyl, nitro, —CH₂—CO—NH₂, heteroaryl, heteroarylalkoxy, heteroaryloxy, cycloalkyl, cycloalkylalkoxy, cycloalkoxy, heterocycloalkyl, heterocycloalkylalkoxy, heterocycloalkoxy, cyano, cyanato, isocyanato, thiocyanato, isothiocyanato, sulfonyl, sulfinyl, sulfonamide, trihalomethanesulfonamido, acyl, acylamino, acyloxy, alkylthio, cycloalkylthio, heterocycloalkylthio, arylthio, heteroarylthio, carboxyl, carbamate or urea, and further wherein the carbocyclyl moiety in said -L-carbocyclyl, the aryl moiety in said -L-aryl, or the heterocyclyl moiety in said -L-heterocyclyl is optionally substituted with one or more groups independently selected from halo, haloalkyl, haloalkoxy, haloaryl, aryl, arylalkoxy, aryloxy, arylalkyl, alkyl, alkenyl, alkynyl, alkoxy, amino, amido, alkylamino, hydroxyl, nitro, —CH₂—CO—NH₂, heteroaryl, heteroarylalkoxy, heteroaryloxy, heteroarylalkyl, cycloalkyl, cycloalkylalkoxy, cycloalkoxy, cycloalkylalkyl, heterocycloalkyl, heterocycloalkylalkoxy, heterocycloalkoxy, heterocycloalkylalkyl, cyano, cyanato, isocyanato, thiocyanato, isothiocyanato, sulfonyl, sulfinyl, sulfonamide, trihalomethanesulfonamido, acyl, acylamino, acyloxy, alkylthio, cycloalkylthio, heterocycloalkylthio, arylthio, heteroarylthio, carboxyl, carbamate or urea.

In formula (I), R³ is chosen from -L-heterocyclyl, -L-carbocyclyl, -L-aryl, —H, or alkoxy, wherein the carbocyclyl moiety in said -L-carbocyclyl, the heterocyclyl moiety in said -L-heterocyclyl or the aryl moiety in said -L-aryl is optionally substituted with one or more groups independently selected from halo, haloalkyl, haloalkoxy, haloaryl, aryl, arylalkoxy, aryloxy, arylalkyl, alkyl, alkenyl, alkynyl, alkoxy, amino, amido, alkylamino, hydroxyl, nitro, —CH₂—CO—NH₂, heteroaryl, heteroarylalkoxy, heteroaryloxy, heteroarylalkyl, cycloalkyl, cycloalkylalkoxy, cycloalkoxy, cycloalkylalkyl, heterocycloalkyl, heterocycloalkylalkoxy, heterocycloalkoxy, heterocycloalkylalkyl, cyano, cyanato, isocyanato, thiocyanato, isothiocyanato, sulfonyl, sulfinyl, sulfonamide, trihalomethanesulfonamido, acyl, acylamino, acyloxy, alkylthio, cycloalkylthio, heterocycloalkylthio, arylthio, heteroarylthio, carboxyl, carbamate or urea. It is preferred that R³ is -L-heterocyclyl, particularly -L-heterocyclyl wherein the heterocyclyl moiety is a saturated heterocyclic ring, and more preferably it is preferred that L is a covalent bond.

Each L is independently selected from —(CH₂)ₙ—(CH₂)ₙ—, —(CH₂)ₙC(═O)(CH₂)ₙ—, —(CH₂)ₙC(═O)NH(CH₂)ₙ—, —(CH₂)ₙNHC(═O)O(CH₂)ₙ—, —(CH₂)ₙNHC(═O)NH(CH₂)ₙ—, —(CH₂)ₙNHC(═S)S(CH₂)ₙ—, —(CH₂)ₙOC(═O)S(CH₂)ₙ—, —(CH₂)ₙNH(CH₂)ₙ—, —(CH₂)ₙO(CH₂)ₙ—, —(CH₂)ₙS(CH₂)ₙ—, and —(CH₂)ₙNHC(═S)NH(CH₂)ₙ—, and each n is independently chosen from 0, 1, 2, 3, 4, 5, 6, 7, and 8. Preferably, in R¹ and R² each L is independently —(CH₂)₁₋₆—, more preferably —(CH₂)₁₋₄—, and even more preferably —CH₂—. Preferably, in R³ L is bond.

L² is C₁₋₁₂ alkylene which is optionally interrupted by one or more (e.g., one, two, three or four) groups independently selected from —O—, —S—, —NH—, —N(alkyl)-, —CO—, —CO—NH— or —CO—N(alkyl)-, or L² is a covalent bond. Preferably, L² is —CH₂—(C₁₋₆ alkylene), —CH₂—CO— or a covalent bond, wherein the alkylene moiety in said —CH₂—(C₁₋₆ alkylene) is optionally interrupted by one or more (e.g., one, two or three) groups independently selected from —O—, —S—, —NH—, —N(alkyl)-, —CO—, —CO—NH—, —CO—N(alkyl)-. More preferably, L² is —(CH₂)₁₋₄—, —CH₂—CO— or a covalent bond. Even more preferably, L² is —(CH₂)₂—, —CH₂—CO— or a covalent bond.

In one preferred embodiment, B is -L²-cyclyl, wherein the cyclyl moiety in said -L²-cyclyl is optionally substituted with one or more groups independently selected from halo, haloalkyl, haloalkoxy, haloaryl, aryl, arylalkoxy, aryloxy, arylalkyl, alkyl, alkenyl, alkynyl, alkoxy, amino, amido, alkylamino, hydroxyl, nitro, —CH₂—CO—NH₂, heteroaryl, heteroarylalkoxy, heteroaryloxy, heteroarylalkyl, cycloalkyl, cycloalkylalkoxy, cycloalkoxy, cycloalkylalkyl, heterocycloalkyl, heterocycloalkylalkoxy, heterocycloalkoxy, heterocycloalkylalkyl, cyano, cyanato, isocyanato, thiocyanato, isothiocyanato, sulfonyl, sulfinyl, sulfonamide, trihalomethanesulfonamido, acyl, acylamino, acyloxy, alkylthio, cycloalkylthio, heterocycloalkylthio, arylthio, heteroarylthio, carboxyl, carbamate or urea.

In another preferred embodiment, B is —(CH₂)₀₋₅-heteroaryl, —(CH₂)₀₋₅-heterocycloalkyl, —(CH₂)₁₋₅—CO-heterocycloalkyl, —H, —(CH₂)₁₋₄—CO—NH₂, or —(CH₂)₁₋₄—CO—NR¹R², wherein the heteroaryl moiety comprised in said —(CH₂)₀₋₅-heteroaryl and the heterocycloalkyl moiety comprised in said —(CH₂)₀₋₅-heterocycloalkyl or in said —(CH₂)₁₋₅—CO-heterocycloalkyl is optionally substituted with one or two groups, preferably with one group, independently selected from halo, alkyl, alkoxy, haloalkyl, haloalkoxy, cyano, hydroxyl, amino, alkylamino, aminoalkyl, amido (e.g., —CO—NH₂), —CH₂—CO—NH₂, or sulfonamide.

In a particularly preferred embodiment, B is —(CH₂)₀₋₅-heteroaryl, wherein the heteroaryl moiety comprised in said —(CH₂)₀₋₅-heteroaryl is preferably selected from oxadiazolyl, thiazolyl or pyrimidinyl and, furthermore, is optionally substituted with one group selected from halo, alkyl, alkoxy, haloalkyl, haloalkoxy, cyano, hydroxyl, amino, alkylamino, aminoalkyl, amido (e.g., —CO—NH₂), —CH₂—CO—NH₂, or sulfonamide. In a further particularly preferred embodiment, B is —(CH₂)₀₋₅-heterocycloalkyl, wherein the heterocycloalkyl moiety comprised in said —(CH₂)₀₋₅-heterocycloalkyl is preferably selected from pyrrolidinyl, piperidinyl, piperazinyl, N-methylpiperazinyl or morpholinyl and, furthermore, is optionally substituted with one group selected from halo, alkyl, alkoxy, haloalkyl, haloalkoxy, cyano, hydroxyl, amino, alkylamino, aminoalkyl, amido (e.g., —CO—NH₂), —CH₂—CO—NH₂, or sulfonamide. In a further particularly preferred embodiment, B is —CH₂-oxadiazolyl, wherein the oxadiazolyl moiety comprised in said —CH₂-oxadiazolyl is optionally substituted with one group selected from halo, alkyl, alkoxy, haloalkyl, haloalkoxy, cyano, hydroxyl, amino, alkylamino or aminoalkyl (accordingly, B may, for example, be aminooxadiazolylmethyl, such as 2-amino-1,3,4-oxadiazol-5-ylmethyl or 3-amino-1,2,4-oxadiazol-5-ylmethyl). In a further particularly preferred embodiment, B is —(CH₂)₁₋₅—CO-heterocycloalkyl, wherein the heterocycloalkyl moiety comprised in said —(CH₂)₁₋₅—CO-heterocycloalkyl is preferably selected from pyrrolidinyl, piperidinyl, piperazinyl, N-methylpiperazinyl or morpholinyl and, furthermore, is optionally substituted with one group selected from halo, alkyl, alkoxy, haloalkyl, haloalkoxy, cyano, hydroxyl, amino, alkylamino, aminoalkyl, amido (e.g., —CO—NH₂), —CH₂—CO—NH₂, or sulfonamide. In a further particularly preferred embodiment, B is —H. In a further particularly preferred embodiment, B is -L²-CO—NH₂, preferably —(CH₂)₁₋₄—CO—NH₂, more preferably —CH₂—CO—NH₂. In a further particularly preferred embodiment, B is -L₂-CO—NR¹R², preferably B is —(CH₂)₁₋₄—CO—NR¹R², more preferably —CH₂—CO—NR¹R².

The substituents on the cyclopropane ring, i.e. the groups -(A) and —NR^{a}—B, are preferably in trans configuration. In that case, the 2-cyclylcyclopropan-1-amine compound of formula (I) may have the configuration (1R,2S) or the configuration (1S,2R) at the cyclopropane ring carbon atoms. The present invention specifically relates to the (1R,2S) stereoisomer of the 2-cyclylcyclopropan-1-amine compound of formula (I). The invention also specifically relates to the (1S,2R) stereoisomer of the 2-cyclylcyclopropan-1-amine compound of formula (I).

In one embodiment, the LSD1 inhibitor to be used in the present invention is a 2-cyclylcyclopropan-1-amine compound which is a compound of the following formula (II) or a pharmaceutically acceptable salt thereof:

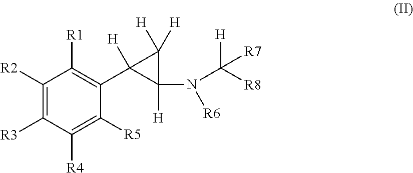

In formula (II), each of R1-R5 is optionally substituted and independently chosen from —H, halo, alkyl, alkoxy, cycloalkoxy, haloalkyl, haloalkoxy, -L-aryl, -L-heteroaryl, -L-heterocyclyl, -L-carbocycle, acylamino, acyloxy, alkylthio, cycloalkylthio, alkynyl, amino, aryl, arylalkyl, arylalkenyl, arylalkynyl, arylalkoxy, aryloxy, arylthio, heteroarylthio, cyano, cyanato, haloaryl, hydroxyl, heteroaryloxy, heteroarylalkoxy, isocyanato, isothiocyanato, nitro, sulfinyl, sulfonyl, sulfonamide, thiocarbonyl, thiocyanato, trihalomethanesulfonamido, O-carbamyl, N-carbamyl, O-thiocarbamyl, N-thiocarbamyl, and C-amido;

R6 is chosen from —H and alkyl;
R7 is chosen from —H, alkyl, and cycloalkyl;
R8 is chosen from —C(═O)NRₓR_{y} and —C(═O)R_{z};
Rₓ when present is chosen from —H, alkyl, alkynyl, alkenyl, -L-carbocycle, -L-aryl, -L-heterocyclyl, all of which are optionally substituted;
R_{y} when present is chosen from —H, alkyl, alkynyl, alkenyl, -L-carbocycle, -L-aryl, -L-heterocyclyl, all of which are optionally substituted;
R_{z} when present is chosen from —H, alkoxy, -L-carbocyclic, -L-heterocyclic, -L-aryl, wherein the aryl, heterocyclyl, or carbocycle is optionally substituted;
each L can be saturated, partially saturated, or unsaturated, and is independently chosen from —(CH₂)ₙ—(CH₂)ₙ—, —(CH₂)ₙC(═O)(CH₂)ₙ—, —(CH₂)ₙC(═O)NH(CH₂)ₙ—, —(CH₂)ₙNHC(═O)O(CH₂)ₙ—, —(CH₂)ₙNHC(═O)NH(CH₂)ₙ—, —(CH₂)ₙNHC(═S)S(CH₂)ₙ—, —(CH₂)ₙOC(═O)S(CH₂)ₙ—, —(CH₂)ₙNH(CH₂)ₙ—, —(CH₂)ₙO(CH₂)ₙ—, —(CH₂)ₙS(CH₂)ₙ—, and —(CH₂)ₙNHC(═S)NH(CH₂)ₙ—, where each n is independently chosen from 0, 1, 2, 3, 4, 5, 6, 7, and 8, wherein optionally substituted refers to zero or 1 to 4 optional substituents independently chosen from acylamino, acyloxy, alkenyl, alkoxy, cycloalkoxy, alkyl, alkylthio, cycloalkylthio, alkynyl, amino, aryl, arylalkyl, arylalkenyl, arylalkynyl, arylalkoxy, aryloxy, arylthio, heteroarylthio, carbocyclyl, cyano, cyanato, halo, haloalkyl, haloaryl, hydroxyl, heteroaryl, heteroaryloxy, heterocyclyl, heteroarylalkoxy, isocyanato, isothiocyanato, nitro, sulfinyl, sulfonyl, sulfonamide, thiocarbonyl, thiocyanato, trihalomethanesulfonamido, O-carbamyl, N-carbamyl, O-thiocarbamyl, N-thiocarbamyl, and C-amido.

In a further embodiment, the LSD1 inhibitor to be used in the present invention is a 2-cyclylcyclopropan-1-amine compound which is a compound of the following formula (III) or a pharmaceutically acceptable salt thereof:

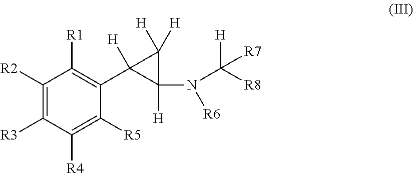

In formula (III), each of R1-R5 is independently chosen from —H, halo, alkyl, alkoxy, cycloalkoxy, haloalkyl, haloalkoxy, -L-aryl, -L-heterocyclyl, -L-carbocyclyl, acylamino, acyloxy, alkylthio, cycloalkylthio, alkynyl, amino, alkylamino, aryl, arylalkyl, arylalkenyl, arylalkynyl, arylalkoxy, aryloxy, arylthio, heteroarylthio, cyano, cyanato, haloaryl, hydroxyl, heteroaryloxy, heteroarylalkoxy, isocyanato, isothiocyanato, nitro, sulfinyl, sulfonyl, sulfonamido, thiocarbonyl, thiocyanato, trihalomethanesulfonamido, O-carbamyl, N-carbamyl, O-thiocarbamyl, N-thiocarbamyl, and C-amido;

R6 is chosen from —H and alkyl;
R7 is chosen from —H, alkyl, and cycloalkyl;
R8 is a -L-heterocyclyl wherein the ring or ring system of said -L-heterocyclyl has from 0-3 substituents chosen from halo, alkyl, alkoxy, cycloalkoxy, haloalkyl, haloalkoxy, -L-aryl, -L-heterocyclyl, -L-carbocyclyl, acylamino, acyloxy, alkylthio, cycloalkylthio, alkynyl, amino, alkylamino, aryl, arylalkyl, arylalkenyl, arylalkynyl, arylalkoxy, aryloxy, arylthio, heteroarylthio, cyano, cyanato, haloaryl, hydroxyl, heteroaryloxy, heteroarylalkoxy, isocyanato, isothiocyanato, nitro, sulfinyl, sulfonyl, sulfonamido, thiocarbonyl, thiocyanato, trihalomethanesulfonamido, O-carbamyl, N-carbamyl, O-thiocarbamyl, N-thiocarbamyl, and C-amido; or
R8 is -L-aryl wherein the ring or ring system of said -L-aryl has from 1-3 substituents chosen from halo, alkyl, alkoxy, cycloalkoxy, haloalkyl, haloalkoxy, -L-aryl, -L-heterocyclyl, -L-carbocyclyl, acylamino, acyloxy, alkylthio, cycloalkylthio, alkynyl, amino, alkylamino, aryl, arylalkyl, arylalkenyl, arylalkynyl, arylalkoxy, aryloxy, arylthio, heteroarylthio, cyano, cyanato, haloaryl, hydroxyl, heteroaryloxy, heteroarylalkoxy, isocyanato, isothiocyanato, nitro, sulfinyl, sulfonyl, sulfonamido, thiocarbonyl, thiocyanato, trihalomethanesulfonamido, O-carbamyl, N-carbamyl, O-thiocarbamyl, N-thiocarbamyl, and C-amido;
each L is independently chosen from —(CH₂)ₙ—(CH₂)ₙ—, —(CH₂)ₙNH(CH₂)ₙ—, —(CH₂)ₙO(CH₂)ₙ—, and —(CH₂)ₙS(CH₂)ₙ—, and where each n is independently chosen from 0, 1, 2, and 3.

In a further embodiment, the LSD1 inhibitor to be used in the present invention is a 2-cyclylcyclopropan-1-amine compound which is a compound of the following formula (IV) or an enantiomer, diastereomer, or mixture thereof, or a pharmaceutically acceptable salt or solvate thereof:

(A′)_{X}-(A)-(B)-(z)-(L)-(D) (IV)

In formula (IV), (A) is heteroaryl or aryl;

each (A′), if present, is independently chosen from aryl, arylalkoxy, arylalkyl, heterocyclyl, aryloxy, halo, alkoxy, haloalkyl, cycloalkyl, haloalkoxy, and cyano, wherein each (A′) is substituted with 0, 1, 2, or 3 substituents independently chosen from halo, haloalkyl, aryl, arylalkoxy, alkyl, alkoxy, cyano, sulfonyl, amido, and sulfinyl;

### X is 0, 1, 2, or 3;

(B) is a cyclopropyl ring, wherein (A) and (Z) are covalently bonded to different carbon atoms of (B);

### (Z) is —NH—;

(L) is chosen from —CH₂CH₂—, —CH₂CH₂CH₂—, and —CH₂CH₂CH₂CH₂—; and
(D) is chosen from —N(—R1)-R2, —O—R3, and —S—R3, wherein:
R1 and R2 are mutually linked to form a heterocyclic ring together with the nitrogen atom that R1 and R2 are attached to, wherein said heterocyclic ring has 0, 1, 2, or 3 substituents independently chosen from —NH₂, —NH(C₁-C₆ alkyl), —N(C₁-C₆ alkyl)(C₁-C₆ alkyl), alkyl, halo, cyano, alkoxy, haloalkyl, and haloalkoxy, or
R1 and R2 are independently chosen from —H, alkyl, cycloalkyl, haloalkyl, and heterocyclyl, wherein the sum of substituents on R1 and R2 together is 0, 1, 2, or 3, and the substituents are independently chosen from —NH₂, —NH(C₁-C₆ alkyl), —N(C₁-C₆ alkyl)(C₁-C₆ alkyl), and fluoro; and
R3 is chosen from —H, alkyl, cycloalkyl, haloalkyl, and heterocyclyl, wherein R3 has 0, 1, 2, or 3 substituents independently chosen from —NH₂, —NH(C₁-C₆ alkyl), —N(C₁-C₆ alkyl)(C₁-C₆ alkyl), and fluoro;
with the proviso that the following compounds are excluded:
N1-[(trans)-2-phenylcyclopropyl]-N2-undecyl-rel-1,2-ethanediamine;
N1-[(trans)-2-phenylcyclopropyl]-N2-tricyclo[3.3.1.13,7]dec-2-yl-rel-1,2-ethanediamine;
N1-cyclooctyl-N2-[(trans)-2-phenylcyclopropyl]-rel-1,2-ethanediamine;
N1,N1-dimethyl-N2-(2-phenylcyclopropyl)-1,3-propanediamine;
N1,N1-dimethyl-N2-(2-phenylcyclopropyl)-1,2-ethanediamine; and
trans-1-phenyl-2-[(2-hydroxyethyl)amino]cyclopropane.

In a further embodiment, the LSD1 inhibitor to be used in the present invention is a 2-cyclylcyclopropan-1-amine compound which is a compound of the following formula (V) or a pharmaceutically acceptable salt or solvate thereof:

(A′)_{X}-(A)-(B)—(Z)-(L)-C(═O)NH₂ (V)

In formula (V), (A) is heteroaryl or aryl;

each (A′), if present, is independently chosen from aryl, arylalkoxy, arylalkyl, heterocyclyl, aryloxy, halo, alkoxy, haloalkyl, cycloalkyl, haloalkoxy, and cyano, wherein each (A′) is substituted with 0, 1, 2 or 3 substituents independently chosen from halo, haloalkyl, aryl, arylalkoxy, alkyl, alkoxy, cyano, sulfonyl, sulfinyl, and carboxamide;

### X is 0, 1, 2, or 3;

(B) is a cyclopropyl ring, wherein (A) and (Z) are covalently bonded to different carbon atoms of (B);

### (Z) is —NH—; and

(L) is —(CH₂)ₘCR₁R₂—, wherein m is 0, 1, 2, 3, 4, 5, or 6, and wherein R₁ and R₂ are each independently hydrogen or C₁-C₆ alkyl;
provided that, if (L) is —CH₂— or —CH(CH₃)—, then X is not 0.

In a further embodiment, the LSD1 inhibitor to be used in the present invention is a 2-cyclylcyclopropan-1-amine compound which is a compound of the following formula (VI) or an enantiomer, a diastereomer, or a mixture thereof, or a pharmaceutically acceptable salt or solvate thereof:

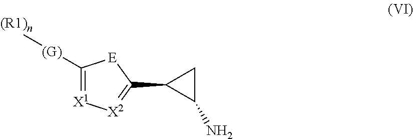

In formula (VI), E is —N(R3)-, —O—, or —S—, or is —X³═X⁴—;

X¹ and X² are independently C(R2) or N;
X³ and X⁴, when present, are independently C(R2) or N;
(G) is a cyclyl group;
each (R1) is independently chosen from alkyl, alkenyl, alkynyl, cyclyl, -L1-cyclyl, -L1-amino, -L1-hydroxyl, amino, amido, nitro, halo, haloalkyl, haloalkoxy, cyano, sulfinyl, sulfonyl, sulfonamide, hydroxyl, alkoxy, urea, carbamate, acyl, or carboxyl;
each (R2) is independently chosen from —H, alkyl, alkenyl, alkynyl, cyclyl, -L1-cyclyl, -L1-amino, -L1-hydroxyl, amino, amido, nitro, halo, haloalkyl, haloalkoxy, cyano, sulfinyl, sulfonyl, sulfonamide, hydroxyl, alkoxy, urea, carbamate, acyl, or carboxyl, wherein each (R2) group has 1, 2, or 3 independently chosen optional substituents or two (R2) groups can be taken together to form a heterocyclyl or aryl group having 1, 2, or 3 independently chosen optional substituents, wherein said optional substituents are independently chosen from alkyl, alkanoyl, heteroalkyl, heterocyclyl, haloalkyl, cycloalkyl, carbocyclyl, arylalkoxy, heterocyclylalkoxy, aryl, aryloxy, heterocyclyloxy, alkoxy, haloalkoxy, oxo, acyloxy, carbonyl, carboxyl, carboxamido, cyano, halogen, hydroxyl, amino, aminoalkyl, amidoalkyl, amido, nitro, thiol, alkylthio, arylthio, sulfonamide, sulfinyl, sulfonyl, urea, or carbamate;
R3 is —H or a (C₁-C₆)alkyl group;
each L1 is independently alkylene or heteroalkylene; and
n is 0, 1, 2, 3, 4 or 5.

In a further embodiment, the LSD1 inhibitor to be used in the present invention is a 2-cyclylcyclopropan-1-amine compound which is a compound of the following formula (VII) or an enantiomer, a diastereomer, or a mixture thereof, or a pharmaceutically acceptable salt or solvate thereof:

(A′)_{X}-(A)-(B)—(Z)-(L)-(D) (VII)

In formula (VII), (A) is heteroaryl or aryl;

each (A′), if present, is independently chosen from aryl, arylalkoxy, arylalkyl, heterocyclyl, aryloxy, halo, alkoxy, haloalkyl, cycloalkyl, haloalkoxy, and cyano, wherein each (A′) is substituted with 0, 1, 2, or 3 substituents independently chosen from halo, haloalkyl, haloalkoxy, aryl, arylalkoxy, alkyl, alkoxy, amido, —CH₂C(═O)NH₂, heteroaryl, cyano, sulfonyl, and sulfinyl;

### X is 0, 1, 2, or 3;

(B) is a cyclopropyl ring, wherein (A) and (Z) are covalently bonded to different carbon atoms of (B);

### (Z) is —NH—;

(L) is chosen from a single bond, —CH₂—, —CH₂CH₂—, —CH₂CH₂CH₂—, and —CH₂CH₂CH₂CH₂—; and
(D) is an aliphatic carbocyclic group or benzocycloalkyl, wherein said aliphatic carbocyclic group or said benzocycloalkyl has 0, 1, 2, or 3 substituents independently chosen from —NH₂, —NH(C₁-C₆ alkyl), —N(C₁-C₆ alkyl)(C₁-C₆ alkyl), alkyl, halo, amido, cyano, alkoxy, haloalkyl, and haloalkoxy;
with the proviso that the following compounds are excluded:
N-(2-phenylcyclopropyl)-cyclopentanamine;
10,11-dihydro-N-(2-phenylcyclopropyl)-5H-dibenzo[a,d]cyclohepten-5-amine; and
trans-N-(2-phenylcyclopropyl)-cyclohexanamine.

In a further embodiment, the LSD1 inhibitor to be used in the present invention is a 2-cyclylcyclopropan-1-amine compound which is a compound of the following formula (VIII) or a pharmaceutically acceptable salt or solvate thereof:

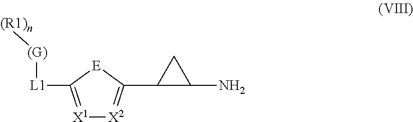

In formula (VIII), E is —X³═X⁴—, —N(R3)-, —S—, or —O—, or;

X¹ and X² are each independently C(R2) or N;
X³ and X⁴, when present, are each independently C(R2) or N;

### L1 is —NH— or —NH—CH₂—;

G is a cyclyl group;
each R1 is independently chosen from alkyl, alkenyl, alkynyl, cyclyl, -L2-cyclyl, -L2-amino, -L2-hydroxyl, amino, amido, nitro, halo, haloalkyl, haloalkoxy, cyano, sulfinyl, sulfonyl, sulfonamide, hydroxyl, alkoxy, urea, carbamate, acyl, or carboxyl;
each R2 is independently chosen from —H, alkyl, alkenyl, alkynyl, cyclyl, -L2-cyclyl, -L2-amino, -L2-hydroxyl, amino, amido, nitro, halo, haloalkyl, haloalkoxy, cyano, sulfinyl, sulfonyl, sulfonamide, hydroxyl, alkoxy, urea, carbamate, acyl, or carboxyl, wherein each R2 group has 1, 2, or 3 independently chosen optional substituents, and further wherein two R2 groups bound to adjacent carbon atoms can be taken together to form a heterocyclyl or aryl group having 1, 2, or 3 independently chosen optional substituents; wherein said optional substituents are each independently chosen from alkyl, alkanoyl, heteroalkyl, heterocyclyl, haloalkyl, cycloalkyl, carbocyclyl, arylalkoxy, heterocyclylalkoxy, aryl, aryloxy, heterocyclyloxy, alkoxy, haloalkoxy, oxo, acyloxy, carbonyl, carboxyl, carboxamido, cyano, halogen, hydroxyl, amino, aminoalkyl, amidoalkyl, amido, nitro, thiol, alkylthio, arylthio, sulfinyl, sulfonyl, sulfonamide, urea or carbamate;
R3 is —H or an (C1-C6)alkyl group;
each L2 is independently chosen from alkylene or heteroalkylene; and
n is 0, 1, 2, 3, 4 or 5.

In a further embodiment, the LSD1 inhibitor to be used in the present invention is a 2-cyclylcyclopropan-1-amine compound which is a compound of the following formula (IX) or a pharmaceutically acceptable salt or solvate thereof:

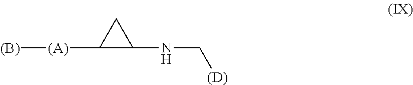

In formula (IX), (A) is a cyclyl group having n substituents (R3);

(B) is a cyclyl group or an -(L1)-cyclyl group, wherein said cyclyl group or the cyclyl moiety comprised in said -(L1)-cyclyl group has n substituents (R2);
(L1) is —O—, —NH—, —N(alkyl)-, alkylene or heteroalkylene;
(D) is a heteroaryl group or an -(L2)-heteroaryl group, wherein said heteroaryl group or the heteroaryl moiety comprised in said -(L2)-heteroaryl group has one substituent (R1), and further wherein said heteroaryl group is covalently bonded to the remainder of the molecule through a ring carbon atom or the heteroaryl moiety comprised in said -(L2)-heteroaryl group is covalently bonded to the (L2) moiety through a ring carbon atom;
(L2) is —O—, —NH—, —N(alkyl)-, alkylene or heteroalkylene;
(R1) is a hydrogen bonding group such as, e.g., —OH, —NH₂, amido, —S(O)₂NH₂, —C(═O)NH₂, —CH₂—C(═O)NH₂, —NH—C(═O)CH₃, —NHCH₃, —N(CH₃)₂ or —CH₂—NH₂;
each (R2) is independently selected from alkyl, alkenyl, alkynyl, cyclyl, amino, amido, C-amido, alkylamino, hydroxyl, nitro, halo, haloalkyl, haloalkoxy, cyano, sulfinyl, sulfonyl, sulfonamide, alkoxy, acyl, carboxyl, carbamate or urea;
each (R3) is independently selected from alkyl, alkenyl, alkynyl, cyclyl, amino, amido, C-amido, alkylamino, hydroxyl, nitro, halo, haloalkyl, haloalkoxy, cyano, sulfinyl, sulfonyl, sulfonamide, alkoxy, acyl, carboxyl, carbamate, or urea; and
n is independently 0, 1, 2, 3 or 4.

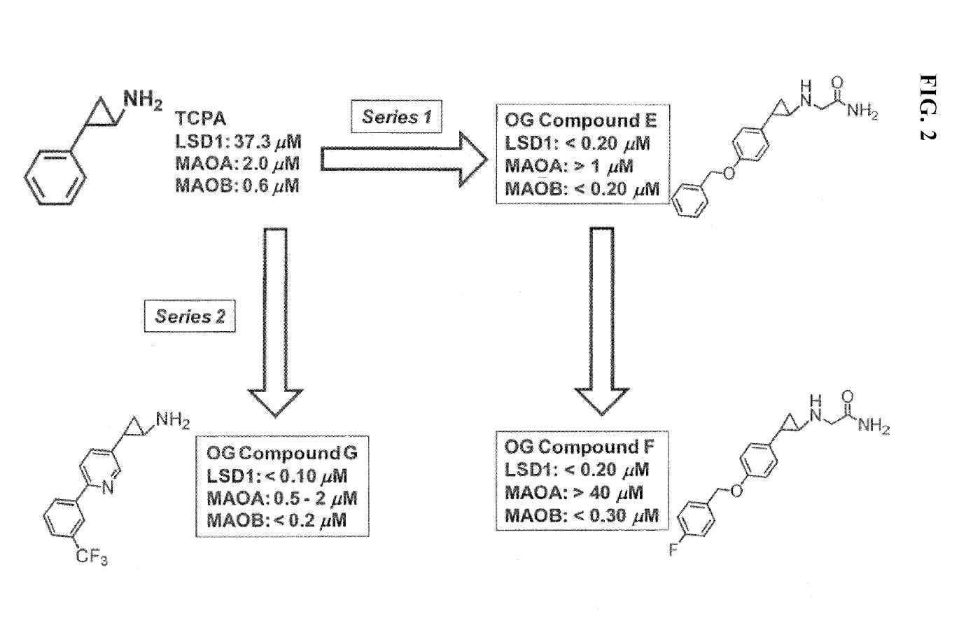

Exemplary non-limiting LSD1 inhibitors are OG Compounds A, B, C and D as shown in FIG. 1, OG Compounds E, F and G as shown in FIG. 2 and Compounds 1 to 10 shown in the Examples, as well as pharmaceutically acceptable salts or solvates thereof.

The 2-cyclylcyclopropan-1-amine compounds disclosed and described herein, including, e.g., the compounds of formulae (I) to (IX), can be prepared by methods known in the art of synthetic chemistry. For example, these compounds can be prepared in accordance with or in analogy to the methods described in WO2010/043721, WO2010/084160, WO2011/035941, WO2011/042217, WO2011/131697, WO2012/013727, and WO2012/013728.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

As used herein, the term “aryl,” refers a carbocyclic aromatic system containing one ring, or two or three rings fused together where in the ring atoms are all carbon. The term “aryl” group includes, but is not limited to groups such as phenyl, naphthyl, or anthracenyl. A preferred aryl group is phenyl.

As used herein, the term “heterocyclyl” or “heterocycle,” each refer to a saturated, partially unsaturated, or fully unsaturated monocyclic, bicyclic, or tricyclic heterocyclic group containing at least one heteroatom as a ring member, wherein each said heteroatom may be independently selected from the group consisting of nitrogen, oxygen, and sulfur wherein the nitron or sulfur atoms may be oxidized (e.g., —N═O, —S(═O)—, or —S(═O)₂—). Additionally, 1, 2, or 3 of the carbon atoms of the heterocyclyl may be optionally oxidized (e.g., to give an oxo group or ═O). One group of heterocyclyls has from 1 to 4 heteroatoms as ring members.

Another group of heterocyclyls has from 1 to 2 heteroatoms as ring members. One group of heterocyclyls has from 3 to 8 ring members in each ring. Yet another group of heterocyclyls has from 3 to 7 ring members in each ring. Again another group of heterocyclyls has from 5 to 6 ring members in each ring. “Heterocyclyl” is intended to encompass a heterocyclyl group fused to a carbocyclyl or benzo ring systems. Examples of heterocyclyl groups include, but are not limited to, pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, homopiperazinyl, azetidinyl, oxetanyl, thietanyl, homopiperidinyl, oxepanyl, thiepanyl, oxazepinyl, diazepinyl, thiazepinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinylimidazolinyl, or imidazolidinyl. Examples of heteroaryls that are heterocyclyls include, but are not limited to, pyridinyl, imidazolyl, imidazopyridinyl, pyrimidinyl, pyrazolyl, triazolyl, pyrazinyl, tetrazolyl, furyl, thienyl, isoxazolyl, thiazolyl, oxadiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, triazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, or furopyridinyl.

As used herein, the term “heteroaryl,” refers to a 3 to 7 membered unsaturated monocyclic ring, or a fused bicyclic, or tricyclic ring system in which the rings are aromatic and in which at least one ring contains at least one atom selected from the group consisting of O, S, and N. One group of heteroaryls has from 5 to 7 ring atoms. Examples of heteroaryl groups include, but are not limited to, pyridinyl, imidazolyl, imidazopyridinyl, pyrimidinyl, pyrazolyl, triazolyl, pyrazinyl, tetrazolyl, furyl, thienyl, isoxazolyl, thiazolyl, oxadiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, triazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, or furopyridinyl.

As used herein, the term “acyl,” refers to a carbonyl attached to an alkenyl, alkyl, aryl, cycloalkyl, heteroaryl, heterocyclyl, or any other moiety where the atom attached to the carbonyl is carbon. An “acetyl” group refers to a —C(═O)CH₃ group. An “alkylcarbonyl” or “alkanoyl” group refers to an alkyl group attached to the parent molecular moiety through a carbonyl group. Examples of such groups include, but are not limited to, methylcarbonyl or ethylcarbonyl. Examples of acyl groups include, but are not limited to, formyl, alkanoyl or aroyl.

As used herein, the term “alkenyl,” refers to a straight-chain or branched-chain hydrocarbon group having one or more double bonds and containing from 2 to 20 carbon atoms. Exemplary alkenyl groups may have from 2 to 6 carbon atoms. A (C2-C6)alkenyl has from 2 to 6 carbon atoms.

As used herein, the term “alkoxy,” refers to an alkyl ether group, wherein the term alkyl is as defined below. Exemplary alkoxy groups may have from 1 to 6 carbon atoms. Examples of suitable alkyl ether groups include, but are not limited to, methoxy, ethoxy, n-propoxy, isopropoxy, n-butoxy, iso-butoxy, sec-butoxy, tert-butoxy, or n-pentoxy.

As used herein, the term “alkyl,” refers to a straight-chain or branched-chain alkyl group containing from 1 to 20 carbon atoms. Exemplary alkyl groups may have from 1 to 10 or, in particular, from 1 to 6 carbon atoms. A (C1-C10)alkyl has from 1 to 10 carbon atoms and a (C1-C6)alkyl has from 1 to 6 carbon atoms and a (C1-C4)alkyl has from 1 to 4 carbon atoms. Examples of alkyl groups include, but are not limited to, methyl, ethyl, n-propyl, isopropyl, n-butyl, isobutyl, sec-butyl, tert-butyl, pentyl, isopentyl, neo-pentyl, iso-amyl, hexyl, heptyl, octyl, or nonyl.

As used herein, the term “alkylene” refers to an alkyl group attached at two positions, i.e. an alkanediyl group. Exemplary alkylene groups may have from 1 to 6 carbon atoms. Examples include, but are not limited to, methylene, ethylene, propylene, butylene, pentylene, hexylene, heptylene, octylene, or nonylene.

As used herein, the term “alkylamino,” refers to an alkyl group attached to the parent molecular moiety through an amino group. Suitable alkylamino groups may be mono- or dialkylated, forming groups including, but not limited to N-methylamino, N-ethylamino, N,N-dimethylamino, N,N-ethylmethylamino, N,N-diethylamino, N-propylamino, and N,N-methylpropylamino.

As used herein, the term “alkynyl,” refers to a straight-chain or branched-chain hydrocarbon group having one or more triple bonds and containing from 2 to 20 carbon atoms. Exemplary alkynyl groups may have from 2 to 6 carbon atoms. A (C2-C6)alkynyl has from 2 to 6 carbon atoms. A (C2-C4)alkynyl has from 2 to 4 carbon atoms. Examples of alkynyl groups include, but are not limited to, ethynyl, propynyl, hydroxypropynyl, butyn-1-yl, butyn-2-yl, pentyn-1-yl, 3-methylbutyn-1-yl, or hexyn-2-yl.

As used herein, the terms “amido” and “carbamoyl,” refer to an amino group as described below attached to the parent molecular moiety through a carbonyl group (e.g., —C(═O)NRR′), or vice versa (—N(R)C(═O)NR′). “Amido” and “carbamoyl” encompass “C-amido”, “N-amido” and “acylamino” as defined herein. R and R′ are as defined herein.

As used herein, the term “C-amido,” refers to a —C(═O)NRR′ group with R and R′ as defined herein.

As used herein, the term “amino,” refers to —NRR′, wherein R and R′ are independently selected from the group consisting of hydrogen, alkyl, heteroalkyl, aryl, carbocyclyl, and heterocyclyl. Additionally, R and R′ may be combined to form a heterocyclyl.

As used herein, the term “arylalkoxy” or “aralkoxy,” refers to an aryl group attached to the parent molecular moiety through an alkoxy group. Examples of arylalkoxy groups include, but are not limited to, benzyloxy or phenethoxy.

As used herein, the term “arylalkyl” or “aralkyl,” refers to an aryl group attached to the parent molecular moiety through an alkyl group.

As used herein, the term “aryloxy,” refers to an aryl group attached to the parent molecular moiety through an oxy (—O—).

As used herein, the term “carbamate,” refers to an O-carbamyl or N-carbamyl group as defined herein.

As used herein, the term “carbonyl,” when alone includes formyl —C(═O)H and in combination is a —C(═O)— group.

As used herein, the term “carboxyl” or “carboxy” refers to —C(═O)OH or the corresponding “carboxylate” anion, such as is in a carboxylic acid salt. An “O-carboxy” group refers to a RC(═O)O— group, where R is as defined herein. A “C-carboxy” group refers to a —C(═O)OR groups where R is as defined herein.

As used herein, the term “cyano” refers to —CN.

As used herein, the term “carbocyclyl” refers to a saturated or partially saturated monocyclic or a fused bicyclic or tricyclic group wherein the ring atoms of the cyclic system are all carbon and wherein each cyclic moiety contains from 3 to 12 carbon atom ring members. “Carbocyclyl” encompasses benzo fused to a carbocyclyl ring system. One group of carbocyclyls have from 5 to 7 carbon atoms. Examples of carbocyclyl groups include, but are not limited to, cyclopropyl, cyclobutyl, cyclopentyl, cyclohexyl, and cycloheptyl, tetrahydronapthyl, indanyl, octahydronaphthyl, 2,3-dihydro-1H-indenyl, or adamantyl.

As used herein, the term “cycloalkyl” refers to a saturated monocyclic, bicyclic or tricyclic group wherein the ring atoms of the cyclic system are all carbon and wherein each cyclic moiety contains from 3 to 12 carbon atom ring members. One group of cycloalkyls has from 5 to 7 carbon atoms. Examples of cycloalkyl groups include, but are not limited to, cyclopropyl, cyclobutyl, cyclopentyl, cyclohexyl, cycloheptyl, or adamantyl.

As used herein, the term “cycloalkenyl” refers to a partially saturated monocyclic, bicyclic or tricyclic group wherein the ring atoms of the cyclic system are all carbon and wherein each cyclic moiety contains from 3 to 12 carbon atom ring members. One group of carboalkenyls have from 5 to 7 carbon atoms. Examples of cycloalkenyl groups include, but are not limited to, cyclobutenyl, cyclopentenyl, or cyclohexenyl.

As used herein, the term “cyclyl” refers to an aryl, heterocyclyl, or carbocyclyl group as defined herein. A “cyclyl” group may, for example, be an aryl group, a cycloalkyl group, a heteroaryl group or a heterocycloalkyl group.

As used herein, the term “halo” or “halogen” refers to fluorine, chlorine, bromine, or iodine.

As used herein, the term “haloalkoxy” refers to a haloalkyl group attached to the parent molecular moiety through an oxygen atom. Examples of haloalkoxy groups include, but are not limited to, trifluoromethoxy, 2-fluoroethoxy, or 3-chloropropoxy.

As used herein, the term “haloalkyl” refers to an alkyl group having the meaning as defined above wherein one or more hydrogens are replaced with a halogen. Specifically embraced are monohaloalkyl, dihaloalkyl or polyhaloalkyl groups. A monohaloalkyl group, for one example, may have an iodo, bromo, chloro or fluoro atom within the group. Dihalo or polyhaloalkyl groups may have two or more of the same halo atoms or a combination of different halo groups. Examples of haloalkyl groups include, but are not limited to, fluoromethyl, difluoromethyl, trifluoromethyl, chloromethyl, dichloromethyl, tri chloromethyl, pentafluoroethyl, heptafluoropropyl, difluorochloromethyl, dichlorofluoromethyl, difluoroethyl, difluoropropyl, dichloroethyl or dichloropropyl.

As used herein, the term “heteroalkyl” refers to a straight or branched alkyl chain, as defined herein above (e.g., an alkyl chain having from 1 to 6 carbon atoms), wherein one, two, or three carbons forming the alkyl chain are each replaced by a heteroatom independently selected from the group consisting of O, N, and S, and wherein the nitrogen and/or sulfur heteroatom(s) (if present) may optionally be oxidized and the nitrogen heteroatom(s) (if present) may optionally be quaternized. The heteroatom(s) O, N and S may, for example, be placed at an interior position of the heteroalkyl group, i.e., the heteroalkyl may be bound to the remainder of the molecule via a carbon atom. Up to two heteroatoms may be consecutive, such as, for example, —CH₂—NH—OCH₃.

As used herein, the term “heteroalkylene” refers to a heteroalkyl group attached at two positions. Examples include, but are not limited to, —CH₂OCH₂—, —CH₂SCH₂—, and —CH₂NHCH₂—, —CH₂S—, or —CH₂NHCH(CH₃)CH₂—.

As used herein, the term “heterocycloalkyl” refers to a heterocyclyl group that is not fully unsaturated e.g., one or more of the rings systems of a heterocycloalkyl is not aromatic. Examples of heterocycloalkyls include piperazinyl, morpholinyl, piperidinyl, or pyrrolidinyl.

As used herein, the term “hydroxyl” or “hydroxy” as used herein, refers to —OH.

As used herein, the term “hydroxyalkyl” as used herein, refers to a hydroxyl group attached to the parent molecular moiety through an alkyl group.

As used herein, the phrase “in the main chain” refers to the longest contiguous or adjacent chain of carbon atoms starting at the point of attachment of a group to the compounds of any one of the formulas disclosed herein.

As used herein, the term phrase “linear chain of atoms” refers to the longest straight chain of atoms independently selected from carbon, nitrogen, oxygen and sulfur.

As used herein, the term “lower” where not otherwise specifically defined, means containing from 1 to and including 6 carbon atoms.

As used herein, the term “lower aryl” means phenyl or naphthyl.

As used herein, the term “lower heteroaryl” means monocyclic heteroaryl comprising five or six ring members, of which between one and four said members may be heteroatoms selected from O, S, or N.

As used herein, the terms “benzo” and “benz” refer to the divalent group C₆H₄=derived from benzene. Examples include, but are not limited to, benzothiophene or benzimidazole.

As used herein, the term “nitro” refers to —NO₂.

As used herein, the terms “sulfonate” “sulfonic acid” and “sulfonic” refers to the —SO₃H group and its anion as the sulfonic acid is used in salt formation.

As used herein, the term “sulfanyl” refers to —S—.

As used herein, the term “sulfinyl” refers to —S(═O)(R)—, with R as defined herein.

As used herein, the term “sulfonyl” refers to —S(═O)₂R, with R as defined herein.

As used herein, the term “sulfonamide” refers to an N-sulfonamido or S-sulfonamido group as defined herein. As used herein, the term “N-sulfonamido” refers to a RS(═O)₂N(R′)— group with R and R′ as defined herein. Exemplary, non-limiting N-sulfonamido groups are —NHSO₂alkyl such as —NHSO₂CH₃, —NHSO₂CH₂CH₃ or —NHSO₂ (isopropyl), and NHSO₂ (optionally substituted aryl) such as —NHSO₂phenyl. As used herein, the term “S-sulfonamido” refers to a —S(═O)₂NRR′, group, with R and R′ as defined herein.

As used herein, the term “urea” refers to a —N(R)C(═O)N(R)(R′) group wherein each R and R′ independently are as defined herein.

As used herein, “hydrogen bonding group” refers to a substituent group, which is capable of taking part in a non-covalent bonding between hydrogen and another atom (usually nitrogen or oxygen). Examples include, but are not limited to, —OH, NH₂, —OH, amido, —S(O)₂NH₂, —C(═O)NH₂, —CH₂—C(═O)NH₂, —NH—C(═O)CH₃, —NHCH₃, —N(CH₃)₂ and —CH₂—NH₂.

As used herein, the term “optionally substituted” means the preceding or anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower cycloalkyl, phenyl, aryl, acyloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxyl, amino, lower alkylamino, arylamino, aminoalkyl, amido, nitro, thiol, lower alkylthio, lower haloalkylthio, lower perhaloalkylthio, arylthio, sulfonate, sulfonic acid, trisubstituted silyl, N₃, SH, SCH₃, C(O)CH₃, CO₂CH₃, CO₂H, pyridinyl, thiophene, furanyl, carbamate, and urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.” In one specific definition, the optional substituents are chosen from hydroxyl, halo, alkyl, alkoxy, haloalkyl, haloalkoxy, —N((C1-C3)alkyl)₂, —NH((C1-C3)alkyl), —NHC(═O)((C1-C3)alkyl), —C(═O)OH, —C(═O)O((C1-C3)alkyl), —C(═O)(C1-C3)alkyl), —C(═O)NH₂, —C(═O)NH(C1-C3)alkyl), —C(═O)NH(cycloalkyl), —C(═O)N((C1-C3)alkyl)₂, —S(═O)₂((C1-C3)alkyl), S(═O)₂NH₂, —S(═O)₂N((C1-C3)alkyl)₂, —S(═O)₂NH((C1-C3)alkyl), —CHF₂, —OCF₃, —OCHF₂, —SCF₃, —CF₃, —CN, —NH₂, —NO₂, or tetrazolyl.

The term R or the term R′, appearing by itself and without a number designation, unless otherwise defined, refers to a moiety selected from the group consisting of hydrogen, alkyl, cycloalkyl, heteroalkyl, aryl, heteroaryl and heterocycloalkyl, Whether an R group has a number designation or not, every R group, including R, R′ and R^{p} where p=(1, 2, 3, . . . p), every substituent, and every term should be understood to be independent of every other in terms of selection from a group. Should any variable, substituent, or term (e.g., aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. Thus, by way of example only, an unsymmetrical group such as —C(═O)N(R)— may be attached to the parent moiety at either the carbon or the nitrogen.

As used herein, the term “2-cyclylcyclopropan-1-amine compound” refers to a compound comprising a 2-cyclylcyclopropan-1-amine moiety or a pharmaceutically acceptable salt or solvate thereof. Exemplary 2-cyclylcyclopropan-1-amine compounds are, without limitation, 2-arylcyclopropan-1-amine compounds (such as 2-phenylcyclopropan-1-amine compounds) and 2-heteroarylcyclopropan-1-amine compounds (such as 2-pyridinylcyclopropan-1-amine compounds or 2-thiazolylcyclopropan-1-amine compounds).

As used herein, the term “2-arylcyclopropan-1-amine compound” refers to a compound comprising a 2-arylcyclopropan-1-amine moiety or a pharmaceutically acceptable salt or solvate thereof.

As used herein, the term “2-heteroarylcyclopropan-1-amine compound” refers to a compound comprising a 2-heteroarylcyclopropan-1-amine moiety or a pharmaceutically acceptable salt or solvate thereof.

As used herein, the term “2-phenylcyclopropan-1-amine compound” refers to a compound comprising a 2-phenylcyclopropan-1-amine moiety or a pharmaceutically acceptable salt or solvate thereof.

As used herein, the term “2-pyridinylcyclopropan-1-amine compound” refers to a compound comprising a 2-pyridinylcyclopropan-1-amine moiety or a pharmaceutically acceptable salt or solvate thereof.

As used herein, the term “2-thiazolylcyclopropan-1-amine compound” refers to a compound comprising a 2-thiazolylcyclopropan-1-amine moiety or a pharmaceutically acceptable salt or solvate thereof.

As used herein, the term “phenelzine compound” refers to a compound comprising a 2-phenylethylhydrazine moiety or a pharmaceutically acceptable salt or solvate thereof.

As used herein, the term “propargylamine compound” refers to a compound comprising a propargylamine moiety or a pharmaceutically acceptable salt or solvate thereof. An exemplary propargylamine compound is, without limitation, pargyline (N-benzyl-N-methylprop-2-yn-1-amine).

In reference to the substituents referred to above, as the skilled artisan is aware, the appropriate selection of the substituents can be made in view of the disclosure herein to provide LSD1 inhibitors, selective LSD1 inhibitors, and dual LSD1/MAOB inhibitors for use in the methods and compositions of the invention.

Other LSD1 inhibitors for use in the invention include, but are not limited to those e.g., disclosed in R Ueda et al. ((2009) J. Am. Chem. Soc. 131(48):17536-17537); C Binda et al. (J. Am. Chem. Soc. 2010 May 19; 132(19):6827-33). Mimasu et al. ((2010) Biochemistry June 22. [Epub ahead of print] PMID: 20568732 [PubMed—as supplied by publisher], J Culhane et al, J Am Chem Soc 2006, 128, 4536-4537, J Culhane et al J Am Chem Soc 2012, 132, 3164-3176, S. K. Sharma et al. J. Med. Chem., 2010, 53 (14), pp 5197-5212, WO 2011/022489, WO 2008/127734 and WO 2007/021839, all of which are explicitly incorporated herein by reference in their entireties to the extent they are not inconsistent with the instant disclosure.

Other phenylcyclopropylamine derivatives and analogs are found e.g., in Kaiser et al. ((1962) J. Med. Chem. 5:1243-1265); Zirkle et al. ((1962) J. Med. Chem. 1265-1284; U.S. Pat. Nos. 3,365,458; 3,471,522; 3,532,749) and Bolesov et al. ((1974) Zhurnal Organicheskoi Khimii 10:8 1661-1669) and Russian Patent No. 230169 (19681030).

Preferably, the LSD1 inhibitor for use in the invention is a selective LSD1 inhibitor or dual inhibitor of LSD1 and MAOB. In one preferred aspect, the selective LSD1 or dual LSD1/MAOB inhibitor has a molecular weight of less than 700 Daltons. In one preferred aspect, the selective LSD1 or dual LSD1 MAOB inhibitor has a molecular weight of less than 500 Daltons. In one preferred aspect, the selective LSD1 or dual LSD1 MAOB inhibitor has a molecular weight of less than 300 Daltons.

Preferably, the LSD1 inhibitor comprises five or less amide bonds (—NH—C═O). Preferably, the LSD1 inhibitor comprises three or less amide bonds (—NH—C═O).

In one aspect, the LSD1 inhibitor for use in the invention has zero amide bonds.

In one aspect, the selective LSD1 and dual LSD1/MAOB inhibitors for use in the invention desirably inhibit LSD1 and/or MAOB selectively compared to MAOA, thus avoiding deleterious side effects associated with administration to animals, including humans, of MAOA inhibitors. As the inventors have described herein, the selective LSD1 inhibitors and the dual LSD1/MAOB inhibitors can be administered in a such a way to an individual e.g., a mammal or human, to achieve concentration in vivo that are expected to inhibit LSD1 and/or MAO-B while avoiding the toxicity associated with inhibition of MAOA and these concentrations are sufficient enough to improve symptoms associated with myeloproliferative or lymphoproliferative disorders.

The invention provides a pharmaceutical composition for treating hematological cancer comprising a pharmaceutically acceptable carrier and a compound which is an inhibitor of LSD1. Preferably the LSD1 inhibitor is a selective LSD1 inhibitor or a dual LSD1/MAOB inhibitor. The ability of a compound to inhibit LSD1 and/or MAOB and its IC50 values for LSD1, MAO-A and MAO-B can be determined in accordance with the experimental protocol described in Example 1. In one specific embodiment, LSD1 inhibitors for use in the invention are as defined above and are chosen from a phenylcyclopropylamine derivative or analog, a phenelzine derivative or analog, or a propargylamine derivative or analog. In another embodiment, the LSD1 inhibitor for use in the invention is chosen from a 2-cyclylcyclopropan-1-amine compound, a phenelzine compound and a propargylamine compound; more preferably, the LSD1 inhibitor for use in the invention is a 2-cyclylcyclopropan-1-amine compound, preferably a 2-arylcyclopropan-1-amine compound or a 2-heteroarylcyclopropan-1-amine compound, and still more preferably a 2-phenylcyclopropan-1-amine compound, a 2-pyridinylcyclopropan-1-amine compound or a 2-thiazolylcyclopropan-1-amine compound.

The invention provides a pharmaceutical composition for treating a myeloproliferative disorder comprising a pharmaceutically acceptable carrier and a compound which is an inhibitor of LSD1. Preferably the LSD1 inhibitor is a selective LSD1 inhibitor or a dual LSD1/MAOB inhibitor. The ability of a compound to inhibit LSD1 and/or MAOB and its IC50 values for LSD1, MAO-A and MAO-B can be determined in accordance with the experimental protocol described in Example 1. In one specific embodiment, LSD1 inhibitors for use in the invention are as defined above and are chosen from a phenylcyclopropylamine derivative or analog, a phenelzine derivative or analog, or a propargylamine derivative or analog. In another embodiment, the LSD1 inhibitor for use in the invention is chosen from a 2-cyclylcyclopropan-1-amine compound, a phenelzine compound and a propargylamine compound; more preferably, the LSD1 inhibitor for use in the invention is a 2-cyclylcyclopropan-1-amine compound, preferably a 2-arylcyclopropan-1-amine compound or a 2-heteroarylcyclopropan-1-amine compound, and still more preferably a 2-phenylcyclopropan-1-amine compound, a 2-pyridinylcyclopropan-1-amine compound or a 2-thiazolylcyclopropan-1-amine compound.

The invention provides a pharmaceutical composition for treating a lymphoproliferative disorder comprising a pharmaceutically acceptable carrier and a compound which is an inhibitor of LSD1. Preferably the LSD1 inhibitor is a selective LSD1 inhibitor or a dual LSD1/MAOB inhibitor. The ability of a compound to inhibit LSD1 and/or MAOB and its IC50 values for LSD1, MAO-A and MAO-B can be determined in accordance with the experimental protocol described in Example 1. In one specific embodiment, LSD1 inhibitors for use in the invention are as defined above and are chosen from a phenylcyclopropylamine derivative or analog, a phenelzine derivative or analog, or a propargylamine derivative or analog. In another embodiment, the LSD1 inhibitor for use in the invention is chosen from a 2-cyclylcyclopropan-1-amine compound, a phenelzine compound and a propargylamine compound; more preferably, the LSD1 inhibitor for use in the invention is a 2-cyclylcyclopropan-1-amine compound, preferably a 2-arylcyclopropan-1-amine compound or a 2-heteroarylcyclopropan-1-amine compound, and still more preferably a 2-phenylcyclopropan-1-amine compound, a 2-pyridinylcyclopropan-1-amine compound or a 2-thiazolylcyclopropan-1-amine compound.

The invention provides a pharmaceutical composition for treating Philadelphia chromosome positive myeloproliferation comprising a pharmaceutically acceptable carrier and a compound which is a selective inhibitor of LSD1. Preferably, LSD1 selective inhibitors (or selective LSD1 inhibitors) have IC50 values for LSD1 which are at least 2-fold lower than the IC50 value for MAOA and/or MAOB. Even more preferably, LSD1 selective inhibitors have IC50 values for LSD1 which are at least 5-fold lower than the IC50 value for MAOA and/or MAOB. Yet even more preferably, LSD1 selective inhibitors have IC50 values for LSD1 which are at least 10-fold lower than the IC50 value for MAOA and/or MAOB. The ability of a compound to inhibit LSD1 and/or MAOB and its IC50 values for LSD1, MAO-A and MAO-B can be determined in accordance with the experimental protocol described in Example 1. In one specific embodiment, dual selective LSD1 inhibitors for use in the invention are as defined above and are chosen from a phenylcyclopropylamine derivative or analog, a phenelzine derivative or analog, or a propargylamine derivative or analog. In another embodiment, the selective LSD1 inhibitor for use in the invention is chosen from a 2-cyclylcyclopropan-1-amine compound, a phenelzine compound and a propargylamine compound; more preferably, the selective LSD1 inhibitor for use in the invention is a 2-cyclylcyclopropan-1-amine compound, preferably a 2-arylcyclopropan-1-amine compound or a 2-heteroarylcyclopropan-1-amine compound, and still more preferably a 2-phenylcyclopropan-1-amine compound, a 2-pyridinylcyclopropan-1-amine compound or a 2-thiazolylcyclopropan-1-amine compound.

The invention also provides a pharmaceutical composition comprising a pharmaceutically acceptable carrier and a compound which is a dual inhibitor selective for LSD1 and MAOB. Preferably, dual LSD1/MAOB inhibitors have IC50 values for LSD1 and MAOB which are at least 2-fold lower than the IC50 value for MAO-A. Even more preferably, dual LSD1/MAOB inhibitors have IC50 values for LSD1 and MAOB which are at least 5-fold lower than the IC50 value for MAO-A. Yet even more preferably, dual LSD1/MAOB inhibitors have IC50 values for LSD1 and MAOB which are at least 10-fold lower than the IC50 value for MAO-A. The ability of a compound to inhibit LSD1 and/or MAOB and its IC50 values for LSD1, MAO-A and MAO-B can be determined in accordance with the experimental protocol described in Example 1. In one specific embodiment, dual selective LSD1/MAOB inhibitors for use in the invention are as defined above and are chosen from a phenylcyclopropylamine derivative or analog, a phenelzine derivative or analog, or a propargylamine derivative or analog. In another embodiment, the selective LSD1 inhibitor for use in the invention is chosen from a 2-cyclylcyclopropan-1-amine compound, a phenelzine compound and a propargylamine compound; more preferably, the selective LSD1 inhibitor for use in the invention is a 2-cyclylcyclopropan-1-amine compound, preferably a 2-arylcyclopropan-1-amine compound or a 2-heteroarylcyclopropan-1-amine compound, and still more preferably a 2-phenylcyclopropan-1-amine compound, a 2-pyridinylcyclopropan-1-amine compound or a 2-thiazolylcyclopropan-1-amine compound.

Typically, compounds for use as LSD1 inhibitors, selective LSD1 inhibitors or dual inhibitors of LSD1 and MAOB can be effective at an amount of from about 0.01 μg/kg to about 100 mg/kg per day based on total body weight. The active ingredient may be administered at once, or may be divided into a number of smaller doses to be administered at predetermined intervals of time. The suitable dosage unit for humans for each administration can be, e.g., from about 1 μg to about 2000 mg, preferably from about 5 μg to about 1000 mg, and even more preferably from about 0.5 mg to about 500 mg. The active ingredient can be administered orally or by other routes of administration e.g., IP, IV, etc. Preferably, the inhibitor is formulated and delivered in such a way as to achieve concentration in vivo that modulate the target activity e.g., LSD1 and/or MAOB. Thus, in a specific embodiment, the effective amount of compound ranges from 0.05 μg/kg to about 100 mg/kg per day based on total body weight, preferably from 0.05 μg/kg to about 50 mg/kg.

It should be understood that the dosage ranges set forth above are exemplary only and are not intended to limit the scope of this invention unless specified. The therapeutically effective amount for each active compound can vary with factors including but not limited to the activity of the compound used, stability of the active compound in the patient's body, the severity of the conditions to be alleviated, the total weight of the patient treated, the route of administration, the ease of absorption, distribution, and excretion of the active compound by the body, the age and sensitivity of the patient to be treated, and the like, as will be apparent to a skilled artisan. The amount of administration can be adjusted as the various factors change over time.

For oral delivery, the active compounds can be incorporated into a formulation that includes pharmaceutically acceptable carriers such as binders (e.g., gelatin, cellulose, gum tragacanth), excipients (e.g., starch, lactose), lubricants (e.g., magnesium stearate, silicon dioxide), disintegrating agents (e.g., alginate, Primogel, and corn starch), and sweetening or flavoring agents (e.g., glucose, sucrose, saccharin, methyl salicylate, and peppermint). The formulation can be orally delivered in the form of enclosed gelatin capsules or compressed tablets. Capsules and tablets can be prepared in any conventional techniques. The capsules and tablets can also be coated with various coatings known in the art to modify the flavors, tastes, colors, and shapes of the capsules and tablets. In addition, liquid carriers such as fatty oil can also be included in capsules.

Suitable oral formulations can also be in the form of suspension, syrup, chewing gum, wafer, elixir, and the like. If desired, conventional agents for modifying flavors, tastes, colors, and shapes of the special forms can also be included. In addition, for convenient administration by enteral feeding tube in patients unable to swallow, the active compounds can be dissolved in an acceptable lipophilic vegetable oil vehicle such as olive oil, corn oil and safflower oil.

The active compounds can also be administered parenterally in the form of solution or suspension, or in lyophilized form capable of conversion into a solution or suspension form before use. In such formulations, diluents or pharmaceutically acceptable carriers such as sterile water and physiological saline buffer can be used. Other conventional solvents, pH buffers, stabilizers, anti-bacteria agents, surfactants, and antioxidants can all be included. For example, useful components include sodium chloride, acetates, citrates or phosphates buffers, glycerin, dextrose, fixed oils, methyl parabens, polyethylene glycol, propylene glycol, sodium bisulfate, benzyl alcohol, ascorbic acid, and the like. The parenteral formulations can be stored in any conventional containers such as vials and ampoules.

Routes of topical administration include nasal, bucal, mucosal, rectal, or vaginal applications. For topical administration, the active compounds can be formulated into lotions, creams, ointments, gels, powders, pastes, sprays, suspensions, drops and aerosols. Thus, one or more thickening agents, humectants, and stabilizing agents can be included in the formulations. Examples of such agents include, but are not limited to, polyethylene glycol, sorbitol, xanthan gum, petrolatum, beeswax, or mineral oil, lanolin, squalene, and the like. A special form of topical administration is delivery by a transdermal patch. Methods for preparing transdermal patches are disclosed, e.g., in Brown, et al. (1988) Ann. Rev. Med. 39:221-229 which is incorporated herein by reference.

Subcutaneous implantation for sustained release of the active compounds may also be a suitable route of administration. This entails surgical procedures for implanting an active compound in any suitable formulation into a subcutaneous space, e.g., beneath the anterior abdominal wall. See, e.g., Wilson et al. (1984) J. Clin. Psych. 45:242-247. Hydrogels can be used as a carrier for the sustained release of the active compounds. Hydrogels are generally known in the art. They are typically made by crosslinking high molecular weight biocompatible polymers into a network, which swells in water to form a gel like material. Preferably, hydrogels are biodegradable or biosorbable. For purposes of this invention, hydrogels made of polyethylene glycols, collagen, or poly(glycolic-co-L-lactic acid) may be useful. See, e.g., Phillips et al. (1984) J. Pharmaceut. Sci., 73: 1718-1720.

The active compounds can also be conjugated, to a water soluble non-immunogenic non-peptidic high molecular weight polymer to form a polymer conjugate. For example, an active compound is covalently linked to polyethylene glycol to form a conjugate. Typically, such a conjugate exhibits improved solubility, stability, and reduced toxicity and immunogenicity. Thus, when administered to a patient, the active compound in the conjugate can have a longer half-life in the body, and exhibit better efficacy. See generally, Burnham (1994) Am. J. Hosp. Pharm. 15:210-218. PEGylated proteins are currently being used in protein replacement therapies and for other therapeutic uses. For example, PEGylated interferon (PEG-INTRON A®) is clinically used for treating Hepatitis B. PEGylated adenosine deaminase (ADAGEN®) is being used to treat severe combined immunodeficiency disease (SCIDS). PEGylated L-asparaginase (ONCAPSPAR®) is being used to treat acute lymphoblastic leukemia (ALL). It is preferred that the covalent linkage between the polymer and the active compound and/or the polymer itself is hydrolytically degradable under physiological conditions. Such conjugates known as “prodrugs” can readily release the active compound inside the body. Controlled release of an active compound can also be achieved by incorporating the active ingredient into microcapsules, nanocapsules, or hydrogels generally known in the art. Other pharmaceutically acceptable prodrugs of the compounds of this invention include, but are not limited to, esters, carbonates, thiocarbonates, N-acyl derivatives, N-acyloxyalkyl derivatives, quaternary derivatives of tertiary amines, N-Mannich bases, Schiff bases, aminoacid conjugates, phosphate esters, metal salts and sulfonate esters.

Liposomes can also be used as carriers for the active compounds of the present invention. Liposomes are micelles made of various lipids such as cholesterol, phospholipids, fatty acids, and derivatives thereof. Various modified lipids can also be used. Liposomes can reduce the toxicity of the active compounds, and increase their stability. Methods for preparing liposomal suspensions containing active ingredients therein are generally known in the art. See, e.g., U.S. Pat. No. 4,522,811; Prescott, Ed., Methods in Cell Biology, Volume XIV, Academic Press, New York, N.Y. (1976).

The active ingredient can be formulated as a pharmaceutically acceptable salt. A “pharmaceutically acceptable salt” is intended to mean a salt that retains the biological effectiveness of the free acids and bases of the specified compound and that is not biologically or otherwise undesirable. A compound for use in the invention may possess a sufficiently acidic, a sufficiently basic, or both functional groups, and accordingly react with any of a number of inorganic or organic bases, and inorganic and organic acids, to form a pharmaceutically acceptable salt. Exemplary pharmaceutically acceptable salts include those salts prepared by reaction of the compounds of the present invention with a mineral or organic acid or an inorganic base, such as salts including sulfates, pyrosulfates, bisulfates, sulfites, bisulfites, phosphates, monohydrophosphates, dihydrophosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4 dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, gamma-hydroxybutyrates, glycollates, tartrates, methane-sulfonates, propanesulfonates, naphthalene-1-sulfonates, naphthalene-2-sulfonates, or mandelates.

As used herein, a “pharmaceutically acceptable carrier” refers to a non-API (API refers to Active Pharmaceutical Ingredient) substances such as disintegrators, binders, fillers, and lubricants used in formulating pharmaceutical products. They are generally safe for administering to humans according to established governmental standards, including those promulgated by the United States Food and Drug Administration and the European Medical Agency.

The active compounds can also be administered in combination with another active agent that synergistically treats or prevents the same symptoms or is effective for another disease or symptom in the patient treated so long as the other active agent does not interfere with or adversely affect the effects of the active compounds of this invention. Such other active agents include but are not limited to anti-inflammation agents, antiviral agents, antibiotics, antifungal agents, antithrombotic agents, cardiovascular drugs, cholesterol lowering agents, anti-cancer drugs, hypertension drugs, and the like.

As used herein, the term “interferon agent” or “alpha interferon” or “interferon alpha” or “a-interferon” refers to the family of interferon proteins that inhibit viral replication, inhibit cellular proliferation, and modulate immune response. The term “alpha interferon” encompasses a variety of commercially available alpha interferons, including, but not limited to, Roferon A interferon (Hoffman-La Roche, Nutley, N.J.), Berofor alpha 2 (Boehringer Ingelheim Pharmaceutical, Inc., Ridgefield, Conn.), Sumiferon (Sumitomo, Japan), Wellferon interferon alpha-n1 (Glaxo-Wellcome Ltd., London, Great Britain). Alpha interferon 2b currently has the broadest approval throughout the world for use in treating HBV. U.S. Pat. No. 4,530,901 (which is hereby incorporated by reference in its entirety) provides a description of the manufacture of alpha interferon 2b.

As used herein, the term “side effects of interferon treatment” include fatigue, muscle aches, headaches, nausea, vomiting, low-grade fever, weight loss, irritability, depression, mild bone marrow suppression, and hair loss.

As used herein, the term “individual in need of treatment” encompasses individuals who have symptoms of myeloproliferation, those who have been diagnosed with a Philadelphia chromosome positive myeloproliferative disease or disorder.

The examples described herein are intended to illustrate different aspects of the invention by exemplification and are not intended to limit the scope of the claims or invention.

### EXAMPLES

### Example 1

### Biochemical Assays

Compounds for use in the methods of the invention can be identified by their ability to inhibit LSD1. The ability of the compounds of the invention to inhibit LSD1 can be tested as follows. Human recombinant LSD1 protein was purchased from BPS Bioscience Inc. In order to monitor LSD1 enzymatic activity and/or its inhibition rate by our inhibitor(s) of interest, di-methylated H3-K4 peptide (Millipore) was chosen as a substrate. The demethylase activity was estimated, under aerobic conditions, by measuring the release of H₂O₂ produced during the catalytic process, using the Amplex® Red peroxide/peroxidase-coupled assay kit (Invitrogen).

Briefly, a fixed amount of LSD1 was incubated on ice for 15 minutes, in the absence and/or in the presence of various concentrations of inhibitor (e.g., from 0 to 75 μM, depending on the inhibitor strength). Tranylcypromine (Biomol International) was used as a control for inhibition. Within the experiment, each concentration of inhibitor was tested in triplicate. After leaving the enzyme interacting with the inhibitor, 12.5 μM of di-methylated H3-K4 peptide was added to each reaction and the experiment was left for 1 hour at 37° C. in the dark. The enzymatic reactions were set up in a 50 mM sodium phosphate, pH 7.4 buffer. At the end of the incubation, Amplex® Red reagent and horseradish peroxidase (HPR) solution were added to the reaction according to the recommendations provided by the supplier (Invitrogen), and left to incubate for 30 extra minutes at room temperature in the dark. A 1 μM H₂O₂ solution was used as a control of the kit efficiency. The conversion of the Amplex® Red reagent to resorufin due to the presence of H₂O₂ in the assay, was monitored by fluorescence (excitation at 540 nm, emission at 590 nm) using a microplate reader (Infinite 200, Tecan). Arbitrary units were used to measure level of H₂O₂ produced in the absence and/or in the presence of inhibitor.

The maximum demethylase activity of LSD1 was obtained in the absence of inhibitor and corrected for background fluorescence in the absence of LSD1. The Ki (IC50) of each inhibitor was estimated at half of the maximum activity.

Human recombinant monoamine oxidase proteins MAO-A and MAO-B were purchased from Sigma Aldrich. MAOs catalyze the oxidative deamination of primary, secondary and tertiary amines. In order to monitor MAO enzymatic activities and/or their inhibition rate by inhibitor(s) of interest, a fluorescent-based (inhibitor)-screening assay was set up. 3-(2-Aminophenyl)-3-oxopropanamine (kynuramine dihydrobromide, Sigma Aldrich), a non fluorescent compound was chosen as a substrate. Kynuramine is a non-specific substrate for both MAOs activities. While undergoing oxidative deamination by MAO activities, kynuramine is converted into 4-hydroxyquinoline (4-HQ), a resulting fluorescent product.

The monoamine oxidase activity was estimated by measuring the conversion of kynuramine into 4-hydroxyquinoline. Assays were conducted in 96-well black plates with clear bottom (Corning) in a final volume of 100 μL. The assay buffer was 100 mM HEPES, pH 7.5. Each experiment was performed in triplicate within the same experiment.

Briefly, a fixed amount of MAO (0.25 μg for MAO-A and 0.5 μg for MAO-B) was incubated on ice for 15 minutes in the reaction buffer, in the absence and/or in the presence of various concentrations of inhibitor (e.g., from 0 to 50 μM, depending on the inhibitor strength). Tranylcypromine (Biomol International) was used as a control for inhibition.

After leaving the enzyme(s) interacting with the inhibitor, 60 to 90 μM of kynuramine was added to each reaction for MAO-B and MAO-A assay respectively, and the reaction was left for 1 hour at 37° C. in the dark. The oxidative deamination of the substrate was stopped by adding 50 μL (v/v) of NaOH 2N. The conversion of kynuramine to 4-hydroxyquinoline, was monitored by fluorescence (excitation at 320 nm, emission at 360 nm) using a microplate reader (Infinite 200, Tecan). Arbitrary units were used to measure levels of fluorescence produced in the absence and/or in the presence of inhibitor.

The maximum of oxidative deamination activity was obtained by measuring the amount of 4-hydroxyquinoline formed from kynuramine deamination in the absence of inhibitor and corrected for background fluorescence in the absence of MAO enzymes. The Ki (1050) of each inhibitor was determined at Vmax/2.

### Example 2

### LSD1 and LSD1/MAO-B Dual Inhibitors

TABLE 1
Exemplary IC50 values for selected compounds
against LSD1, MAO-A, and MAO-B.

| | LSD1 | | |
|---|---|---|---|
| Compound No. | IC50 (uM) | MAO-A IC50 (uM) | MAO-B IC50 (uM) |
| Compound 1 | <0.20 | >2 | <0.20 |
| Compound 2 | <0.20 | >2 | <0.20 |
| Compound 3 | <0.10 | >2 | >2 |
| Compound 4 | <0.10 | >2 | >2 |
| Compound 5 | <0.20 | >0.5 | >1 |
| Compound 6 | <0.07 | >1 | >1 |
| Compound 7 | <0.07 | >2 | >2 |
| Compound 8 | <0.07 | >1 | >10 |

Compounds 1-8 are phenylcyclopropylamine derivatives or analogs as in WO2010/043721 (PCT/EP2009/063685), WO2010/084160 (PCT/EP2010/050697), PCT/EP2010/055131; PCT/EP2010/055103; and EP applications number EP10171345, EP10187039 and EP10171342.

Compound 1 corresponds to

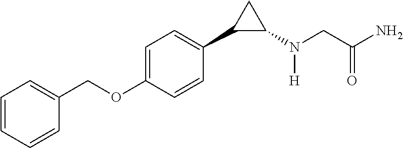

and can be prepared as disclosed in WO 2011/042217.

Compound 2 corresponds to the (1R,2S) isomer of compound 1 and can be prepared following the methods disclosed in WO 2011/042217.

Compound 3 is

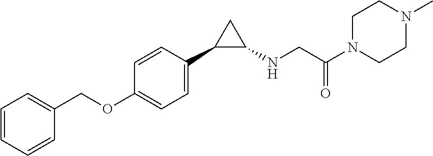 [SMILES: CN1CCN(C(=O)CN[C@H]2C[C@@H]2c2ccc(OCc3ccccc3)cc2)CC1]

and can be prepared as disclosed in WO 2010/043721.

Compound 4 is

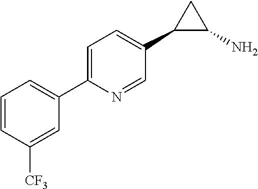 [SMILES: N[C@H]1C[C@@H]1c1ccc(-c2cccc(C(F)(F)F)c2)nc1]

and can be prepared as disclosed in WO 2011/035941.

Compound 5 is

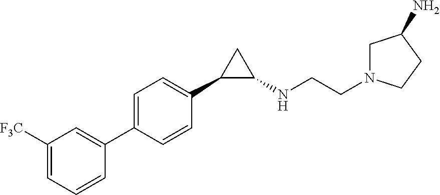 [SMILES: N[C@H]1CCN(CCN[C@H]2C[C@@H]2c2ccc(-c3cccc(C(F)(F)F)c3)cc2)C1]

and can be prepared as disclosed in WO 2012/013727.

Compound 6 is

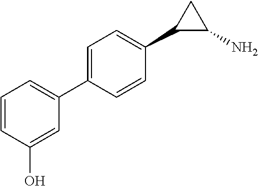

and can be prepared as disclosed in WO 2012/013727.

Compound 7 is

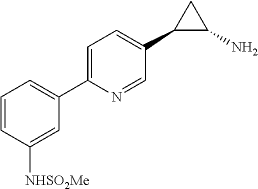

and can be prepared as disclosed in WO 2012/013727.

Compound 8 is

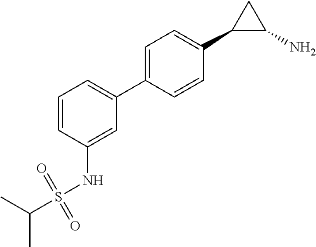 [SMILES: CC(C)S(=O)(=O)Nc1cccc(-c2ccc([C@H]3C[C@@H]3N)cc2)c1]

and can be prepared as disclosed in WO 2012/013727.

### Example 3

### LSD1 and LSD1/MAO-B Dual Inhibitors Increase Histone Lysine Methylation in Cell Based Assays

Histone from SH-SY5Y cells grown in the presence of Compound Dual-1 (a dual LSD1/MAOB inhibitor) (Compound 1 in Example 2 above) or tranylcypromine (parnate) for 1, 2, and 3 days were extracted and subjected to western blot analysis using a commercially available antibody specific for dimethylated H3.K4. B-actin was used as a loading control.

The results of a western blot stained for H3K4 methylation with SH-SY5Y cells grown in the presence of Compound Dual-1 or tranylcypromine (parnate) for 1, 2, and 3 days, showing that this compound, Dual-1, increases H3K4 methylation in cells in a time dependent manner and furthermore Compound Dual-1 appears to be 10-fold or more potent at increasing global dimethylated H3K4 levels as compared to tranylcypromine.

Furthermore, the inventors have conducted similar studies for other dual inhibitors of LSD1/MAOB and with selective LSD1 inhibitors and found that these compounds can increase dimethylated H3K4 levels in similarly performed assays.

### Example 4

### LSD1 Inhibitors can be Administered Safely to Mammals

Maximum tolerated dose studies and pharmacokinetics for several LSD1 inhibitors were assessed to determine if the compound can be administered to mammals safely at doses that are expected to achieve therapeutic effects. Results in chronic dosing experiments indicate that therapeutic levels can be reached in vivo.

### Example 5

### LSD1 Inhibitors Inhibit Platelet Levels in Mammals

### Method for Determination of Effects of LSD1 Inhibitors on Platelets:

3 mice were treated for 5 consecutive days with the compounds and doses indicated in table 2. On the fifth day, 60 min after the administration, mice were sacrificed and blood was collected in sodium citrate-containing tubes for haemogram analysis. Platelet levels were determined and referred as % of platelets compared with the levels found in mice treated with vehicle. Platelet levels were determined in a standard hematology analyzer (Abacus Junior Vet, from Diatron) following the manufacturer's instructions.

20% 2-hydroxypropyl-β-cyclodextrin in H₂O was used as a vehicle. When necessary, 10% DMSO was also added in the vehicle. Each day, compounds were administered in a single intraperitoneal injection with administration volumes of 15 ml/kg.

Mice strain was Hsd:Athymic Nude-Foxn1nu. Animals were maintained in air and temperature controlled cages with regular supply of water and food.

TABLE 2
Results of platelet levels after 5 consecutive once
daily injections of LSD1 inhibitors at the indicated dose

| | Dose | % platelets vs. |
|---|---|---|
| Compound | (mg/kg) | Vehicle |
| Compound 1 | 5 | 91 |
| | 10 | 66 |
| | 20 | 55 |
| | 40 | 34 |
| Compound 2 | 20 | 35 |
| | 40 | 27 |
| | 60 | 16 |
| Compound 3 | 1 | 46 |
| | 3 | 16 |
| | 10 | 5 |
| Compound 4 | 3 | 87 |
| | 9 | 30 |
| | 10 | 19 |
| | 18 | 19 |
| | 30 | 7 |
| | 36 | 11 |
| Compound 5 | 10 | 87 |
| | 30 | 63 |
| Compound 6 | 17 | 56 |
| | 34 | 46 |
| | 52 | 63 |
| Compound 7 | 20 | 68 |
| | 40 | 44 |
| Compound 8 | 30 | 10 |

Compounds 1-8 in Table 2 are the same compounds 1-8 in Example 2.

These results show that LSD1 inhibitors, selective LSD1 inhibitors and dual inhibitors of LSD1 and MAOB reduce platelet levels. These inhibitors can also reduce the levels of other blood cells, as shown below for compound 3:

| | | Dose (mg/kg) | | |
|---|---|---|---|---|
| | Vehicle | 1 | 3 | 10 |
| White Blood Cells | 100 | 86 | 76 | 29 |
| Lymphocytes | 100 | 115 | 90 | 35 |
| Granulocytes | 100 | 69 | 63 | 23 |
| Red blood cells | 100 | 103 | 101 | 92 |

Measurements of all blood cell types were conducted in the same manner as described above for platelets. Data in the table above are expressed as the % of cells vs vehicle.

### Example 6

### In Vitro Cytotoxicity of the Compounds on Hematological Cancer Cell Lines

K562 chronic myelogenous leukemia cells were seeded at different cell densities in 96-well plates. 24 hours later, compounds were added at serial dilutions. 72 h after compound addition, cell viability was determined with a fluorometry-based assay and the concentration at which 50% of the cells remain viable (EC50) was calculated using nonlinear regression.

A table with the calculated EC50 at three different cell densities is included for some compounds.

| EC50 | | | | | | |
|---|---|---|---|---|---|---|
| (μM) | Compound 4 | Compound 6 | Compound 7 | Compound 8 | Compound 9 | Compound 10 |
| cell density A | 3.9 | 16.2 | 19.4 | 17.2 | 31.5 | 14.8 |
| cell density | 3.9 | 10.3 | 20.4 | 16.3 | 57.1 | 13.2 |
| 3 × A | | | | | | |
| cell density | 4.4 | 16.8 | 23.5 | 19.7 | 95.1 | 17.4 |
| 10 × A | | | | | | |

Following the same protocol, in vitro cytotoxicity against other hematological cancer cell lines was tested. Data for compound 8 are provided below:

| | Cell line | Disease | GI₅₀ (uM) |
|---|---|---|---|
| Leukemia | Jurkat | acute T cell leukemia | 18.18 |
| | HL-60 | acute promyelocytic leukemia | 21.15 |
| Multiple | RPMI- | Multiple myeloma | 35.60 |
| Myeloma | 8226 | | |

Compound 9 is

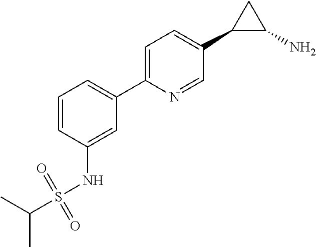 [SMILES: CC(C)S(=O)(=O)Nc1cccc(-c2ccc([C@H]3C[C@@H]3N)cn2)c1]

and can be prepared as described in WO2012/013727.

Compound 10 is

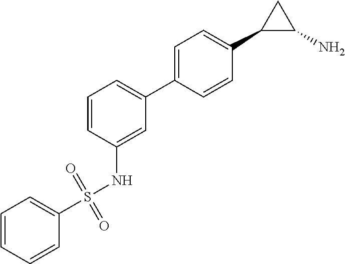 [SMILES: N[C@H]1C[C@@H]1c1ccc(-c2cccc(NS(=O)(=O)c3ccccc3)c2)cc1]

and can be prepared as described in WO2012/013727,

1-2. (canceled)

3. A method of treating or preventing a hematological cancer comprising administering to an individual a therapeutically effective amount of a LSD1 inhibitor.

4. (canceled)

5. The method of claim 3, wherein said hematological cancer is a hematological cancer caused by or related to myeloproliferation.

6. The method of claim 3, wherein said hematological cancer is acute myelogenous leukemia (AML), chronic myelogenous leukemia (CML), chronic neutrophilic leukemia, or chronic eosinophilic leukemia.

7. The method of claim 3, wherein said hematological cancer is a hematological cancer caused by or related to lymphoproliferation.

8. The method of claim 3, wherein said hematological cancer is follicular lymphoma, chronic lymphocytic leukemia (CLL), acute lymphoblastic leukemia (ALL), hairy cell leukemia, lymphoma, multiple myeloma, or Waldenstrom's macroglobulinemia.

9. The method of claim 3, wherein said hematological cancer is a lymphoma chosen from precursor B-lymphoblastic leukemia/lymphoma, B-cell chronic lymphocytic leukemia/small lymphocytic lymphoma, B-cell prolymphocytic leukemia, lymphoplasmacytic lymphoma, splenic marginal zone B-cell lymphoma (+/− villous lymphocytes), hairy cell leukemia, plasma cell myeloma/plasmacytoma, extranodal marginal zone B-cell lymphoma of mucosa-associated lymphoid tissue type, nodal marginal zone lymphoma (+/− monocytoid B-cells), follicle center lymphoma, follicular, mantle cell lymphoma, diffuse large cell B-cell lymphoma (mediastinal large B-cell lymphoma or primary effusion lymphoma), Burkitt's lymphoma/Burkitt's cell leukemia, precursor T-lymphoblastic lymphoma/leukemia, T cell prolymphocytic leukemia, T-cell granular lymphocytic leukemia, aggressive NK-Cell leukemia, adult T cell lymphoma/leukemia (HTLV1+), extranodal NK/T-cell lymphoma (nasal type), enteropathy-type T-cell lymphoma, hepatosplenic gamma-delta T-cell lymphoma, subcutaneous panniculitis-like T-cell lymphoma, mycosis fungoides/Sézary's syndrome, anaplastic large cell lymphoma (T/null cell, primary cutaneous type), peripheral T cell lymphoma (not otherwise characterized), angioimmunoblastic T cell lymphoma, anaplastic large cell lymphoma (T/null cell, primary systemic type), nodular lymphocyte predominance Hodgkin's lymphoma, or classical Hodgkin's lymphoma (nodular sclerosis Hodgkin's lymphoma, lymphocyte-rich classical Hodgkin's lymphoma, mixed cellularity Hodgkin's lymphoma or lymphocyte depletion Hodgkin's lymphoma).

10. The method of claim 3, wherein said hematological cancer is multiple myeloma.

11. The method of claim 3, wherein said hematological cancer is CML, AML, or ALL.

12. (canceled)

13. The method of claim 3, wherein said LSD1 inhibitor is a selective LSD1 inhibitor.

14-16. (canceled)

17. The method of claim 3, wherein said LSD1 inhibitor is an arylcyclopropan-1-amine compound or a 2-heteroarylcyclopropan-1-amine compound.

18-19. (canceled)

20. The method of claim 3, wherein said LSD1 inhibitor is a 2-cyclylcyclopropan-1-amine compound of formula (I) or an enantiomer, a diastereomer or a mixture of stereoisomers (including a racemic mixture or diastereomer mixture) thereof, or a pharmaceutically acceptable salt or solvate thereof: 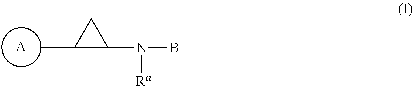
wherein:
A is cyclyl optionally having 1, 2, 3, or 4 substituents A′;
each A′ is independently selected from -L¹-cyclyl, alkyl, alkenyl, alkynyl, alkoxy, amino, amido, —CH₂—CO—NH₂, alkylamino, hydroxyl, nitro, halo, haloalkyl, haloalkoxy, cyano, sulfonyl, sulfinyl, sulfonamide, acyl, carboxyl, carbamate and urea, wherein the cyclyl moiety comprised in said -L¹-cyclyl is optionally further substituted with one or more groups independently selected from halo, haloalkyl, haloalkoxy, aryl, arylalkoxy, aryloxy, arylalkyl, alkyl, alkenyl, alkynyl, alkoxy, amino, amido, alkylamino, hydroxyl, nitro, —CH₂—CO—NH₂, heteroaryl, heteroarylalkoxy, heteroaryloxy, heteroarylalkyl, cyano, sulfonyl, sulfinyl, sulfonamide, acyl, carboxyl, carbamate and urea;
each L¹ is independently selected from a covalent bond, —(CH₂)₁₋₆—, —(CH₂)₀₋₃—O—(CH₂)₀₋₃—, —(CH₂)₀₋₃—NH—(CH₂)₀₋₃— and —(CH₂)₀₋₃—S—(CH₂)₀₋₃—;
B is -L²-cyclyl, —H, -L²-CO—NH₂, -L²-CO—NR¹R² or -L²-CO—R³, wherein the cyclyl moiety in said -L²-cyclyl is optionally substituted with one or more groups independently selected from halo, haloalkyl, haloalkoxy, haloaryl, aryl, arylalkoxy, aryloxy, arylalkyl, alkyl, alkenyl, alkynyl, alkoxy, amino, amido, alkylamino, hydroxyl, nitro, —CH₂—CO—NH₂, heteroaryl, heteroarylalkoxy, heteroaryloxy, heteroarylalkyl, cycloalkyl, cycloalkylalkoxy, cycloalkoxy, cycloalkylalkyl, heterocycloalkyl, heterocycloalkylalkoxy, heterocycloalkoxy, heterocycloalkylalkyl, cyano, cyanato, isocyanato, thiocyanato, isothiocyanato, sulfonyl, sulfinyl, sulfonamide, trihalomethanesulfonamido, acyl, acylamino, acyloxy, alkylthio, cycloalkylthio, heterocycloalkylthio, arylthio, heteroarylthio, carboxyl, carbamate and urea;
R^{a} is —H or alkyl;
R¹ and R² are each independently selected from —H, alkyl, alkynyl, alkenyl, -L-carbocyclyl, -L-aryl, and -L-heterocyclyl, wherein said alkyl, said alkynyl or said alkenyl is optionally substituted with one or more groups independently selected from halo, haloalkoxy, haloaryl, aryl, arylalkoxy, aryloxy, alkoxy, amino, amido, alkylamino, hydroxyl, nitro, —CH₂—CO—NH₂, heteroaryl, heteroarylalkoxy, heteroaryloxy, cycloalkyl, cycloalkylalkoxy, cycloalkoxy, heterocycloalkyl, heterocycloalkylalkoxy, heterocycloalkoxy, cyano, cyanato, isocyanato, thiocyanato, isothiocyanato, sulfonyl, sulfinyl, sulfonamide, trihalomethanesulfonamido, acyl, acylamino, acyloxy, alkylthio, cycloalkylthio, heterocycloalkylthio, arylthio, heteroarylthio, carboxyl, carbamate and urea, and further wherein the carbocyclyl moiety in said -L-carbocyclyl, the aryl moiety in said -L-aryl, or the heterocyclyl moiety in said -L-heterocyclyl is optionally substituted with one or more groups independently selected from halo, haloalkyl, haloalkoxy, haloaryl, aryl, arylalkoxy, aryloxy, arylalkyl, alkyl, alkenyl, alkynyl, alkoxy, amino, amido, alkylamino, hydroxyl, nitro, —CH₂—CO—NH₂, heteroaryl, heteroarylalkoxy, heteroaryloxy, heteroarylalkyl, cycloalkyl, cycloalkylalkoxy, cycloalkoxy, cycloalkylalkyl, heterocycloalkyl, heterocycloalkylalkoxy, heterocycloalkoxy, heterocycloalkylalkyl, cyano, cyanato, isocyanato, thiocyanato, isothiocyanato, sulfonyl, sulfinyl, sulfonamide, trihalomethanesulfonamido, acyl, acylamino, acyloxy, alkylthio, cycloalkylthio, heterocycloalkylthio, arylthio, heteroarylthio, carboxyl, carbamate and urea;
R³ is selected from -L-heterocyclyl, -L-carbocyclyl, -L-aryl, —H, and alkoxy, wherein the carbocyclyl moiety in said -L-carbocyclyl, the heterocyclyl moiety in said -L-heterocyclyl or the aryl moiety in said -L-aryl is optionally substituted with one or more groups independently selected from halo, haloalkyl, haloalkoxy, haloaryl, aryl, arylalkoxy, aryloxy, arylalkyl, alkyl, alkenyl, alkynyl, alkoxy, amino, amido, alkylamino, hydroxyl, nitro, —CH₂—CO—NH₂, heteroaryl, heteroarylalkoxy, heteroaryloxy, heteroarylalkyl, cycloalkyl, cycloalkylalkoxy, cycloalkoxy, cycloalkylalkyl, heterocycloalkyl, heterocycloalkylalkoxy, heterocycloalkoxy, heterocycloalkylalkyl, cyano, cyanato, isocyanato, thiocyanato, isothiocyanato, sulfonyl, sulfinyl, sulfonamide, trihalomethanesulfonamido, acyl, acylamino, acyloxy, alkylthio, cycloalkylthio, heterocycloalkylthio, arylthio, heteroarylthio, carboxyl, carbamate and urea;
each L is independently selected from —(CH2)n-(CH2)n-, —(CH2)nC(═O)(CH2)n-, —(CH2)nC(═O)NH(CH2)n-, —(CH2)nNHC(═O)O(CH2)n-, —(CH2)nNHC(═O)NH(CH2)n-, —(CH2)nNHC(═S)S(CH2)n-, —(CH2)nOC(═O)S(CH2)n-, —(CH2)nNH(CH2)n-, —(CH2)nO(CH2)n-, —(CH2)nS(CH2)n-, and —(CH2)nNHC(═S)NH(CH2)n-, wherein each n is independently selected from 0, 1, 2, 3, 4, 5, 6, 7, and 8; and
L2 is C1-12 alkylene which is optionally interrupted by one or more groups independently selected from —O—, —S—, —NH—, —N(alkyl)-, —CO—, —CO—NH— and —CO—N(alkyl)-, or L2 is a covalent bond.

21-22. (canceled)

23. The method of claim 20, wherein A is aryl or heteroaryl which is unsubstituted or has 1 or 2 substituents A′.

24. The method of claim 23 wherein A is phenyl, pyridinyl, pyrimidinyl, thiophenyl, benzothiophenyl, pyrrolyl, indolyl, furanyl, or thiazolyl, and wherein A is unsubstituted or has 1 or 2 substituents A′.

25-29. (canceled)

30. The method of claim 20, wherein A is aryl or heteroaryl, and further wherein said aryl or said heteroaryl optionally has one substituent A′ selected from -L¹-aryl, -L¹-cycloalkyl, -L¹-heteroaryl and -L¹-heterocycloalkyl, wherein the aryl moiety in said -L¹-aryl, the cycloalkyl moiety in said -L¹-cycloalkyl, the heteroaryl moiety in said -L¹-heteroaryl or the heterocycloalkyl moiety in said -L¹-heterocycloalkyl is optionally substituted with halo, haloalkyl, hydroxy, N-sulfonamido or cyan; and wherein L¹ is a covalent bond, —CH₂—, —O—, —O—CH₂—, —O—(CH₂)₂—, —NH— or —NH—CH₂—.

31. (canceled)

32. The method of claim 20 wherein A is phenyl, pyridinyl, or thiazolyl, and wherein A optionally has one substituent A′ selected from phenyl, —CH₂-phenyl, and —O—CH₂-phenyl, wherein said phenyl, the phenyl moiety in said —CH₂-phenyl, or the phenyl moiety in said —O—CH₂-phenyl is optionally substituted with halo, haloalkyl, hydroxy, N-sulfonamido or cyano.

33. (canceled)

34. The method of claim 20, wherein B is -L²-cyclyl, and further wherein the cyclyl moiety in said -L²-cyclyl is optionally substituted with one or more groups independently selected from halo, haloalkyl, haloalkoxy, haloaryl, aryl, arylalkoxy, aryloxy, arylalkyl, alkyl, alkenyl, alkynyl, alkoxy, amino, amido, alkylamino, hydroxyl, nitro, —CH₂—CO—NH₂, heteroaryl, heteroarylalkoxy, heteroaryloxy, heteroarylalkyl, cycloalkyl, cycloalkylalkoxy, cycloalkoxy, cycloalkylalkyl, heterocycloalkyl, heterocycloalkylalkoxy, heterocycloalkoxy, heterocycloalkylalkyl, cyano, cyanato, isocyanato, thiocyanato, isothiocyanato, sulfonyl, sulfinyl, sulfonamide, trihalomethanesulfonamido, acyl, acylamino, acyloxy, alkylthio, cycloalkylthio, heterocycloalkylthio, arylthio, heteroarylthio, carboxyl, carbamate and urea.

35-36. (canceled)

37. The method of claim 34, wherein the cyclyl moiety in said -L²-cyclyl is aryl or cycloalkyl.

38. The method of claim 34 wherein the cyclyl moiety in said -L²-cyclyl is heteroaryl or heterocycloalkyl.

39. The method of claim 38, wherein the cyclyl moiety in said -L²-cyclyl is oxadiazolyl, thiazolyl, or pyrimidinyl, pyrrolidinyl, piperidinyl, piperazinyl, N-methylpiperazinyl, or morpholinyl.

40-41. (canceled)

42. The method of claim 34, wherein L² is —(CH₂)₁₋₄—, —CH₂—CO—, or a covalent bond.

43-50. (canceled)

51. The method of claim 20, wherein B is -L²-CO—NH₂, —(CH₂)₁₋₄—CO—NH₂, -L²-CO—NR¹R², —(CH₂)₁₋₄—CO—NR¹R², -L²-CO—R³, or —(CH₂)₁₋₄—CO—R³.

52-59. (canceled)

60. The method of claim 20, wherein the substituents on the cyclopropane ring are in trans configuration.

61-66. (canceled)

67. The method of claim 3, wherein said LSD1 inhibitor is to be administered in combination with one or more further therapeutic agents.

68-73. (canceled)

74. The method of claim 3, wherein said individual is a human.
